(19) United States
(12) Patent Application Publication
(10) Pub. No.: US 2024/0374857 A1
(43) Pub. Date: Nov. 14, 2024

(54) A VALVE UNIT FOR A RESPIRATORY SUPPORT SYSTEM AND AN ASSEMBLY THEREFOR

(71) Applicant: Fisher & Paykel Healthcare Limited, Auckland (NZ)

(72) Inventors: Tristan Charles Ian WILSON, Auckland (NZ); Alexander Edwin MACKENZIE, Auckland (NZ)

(21) Appl. No.: 18/685,877

(22) PCT Filed: Aug. 23, 2022
(86) PCT No.: PCT/NZ2022/050108

§ 371 Date: Feb. 22, 2024

(30) Foreign Application Priority Data
Aug. 24, 2021 (AU) 2021221545

(51) Int. Cl. A61M 16/16; A61M 39/24

(52) U.S. Cl. CPC A61M 16/164; A61M 39/24; A61M 2039/2433

(57) ABSTRACT

A valve unit (10) for a fluid supply chamber (20) of a respiratory support system (100) that provides humidified respiratory gases is provided. The valve unit comprises a first port (11) and a second port (12) and a fluid passageway (14) formed therebetween. The valve unit further comprises a valve (13) arranged in the fluid passageway (14) for controlling the flow of fluid between the first port (11) and second port (12). The valve unit is arranged to seal against an interior surface (21) of the fluid supply chamber (20) proximate the first port (11) and against an exterior surface (22) of the fluid supply chamber (20) proximate the second port (12) when the 10 valve unit (10) is connected to the fluid supply chamber (20).

### TECHNICAL FIELD

The present disclosure generally relates to respiratory support systems. More particularly, the present disclosure relates to accessories for use with a respiratory support system providing humidified respiratory gases to a user.

### BACKGROUND

Some conventional respiratory support systems comprise a humidification chamber that can be connected to an auxiliary fluid supply chamber. The humidification chamber contains water used to humidify the gases flow flowing through the humidification chamber. Sometimes the volume of water that the humidification chamber can hold is not sufficient for the entire therapy session, whereby the water may run out before the therapy session ends. By connecting an auxiliary fluid supply chamber, e.g. a water bottle, to the humidification chamber, water run out could be prevented.

Some conventional auxiliary fluid supply chambers comprise a spike port that upon engagement with a spike connector allows water to be released from the auxiliary fluid supply chamber. The humidification chamber may have a permanently attached tube that has a spike connector on the other end. To access the fluid of the auxiliary fluid supply chamber this spike connector needs to be inserted into the spike port of the auxiliary fluid supply chamber. The spike port typically comprises a membrane that is required to be pierced in order to allow water to flow from the fluid supply chamber to the humidification chamber. However, piercing the membrane may be associated with a risk of damaging the spike port possibly with undesired leakage following as a result.

A fluid supply chamber solution which overcomes problems of the prior art or provides the public with a useful choice would thus be advantageous.

### SUMMARY

The present disclosure relates to a valve unit (hereinafter also referred to as a “bottle spike port”) and associated fluid supply chamber to enabling fluid to flow from the fluid supply chamber to a humidification chamber of a respiratory support system and which overcomes problems or provides the public with a useful choice.

In a first aspect there may be provided a valve unit for a fluid supply chamber of a respiratory support system that provides humidified respiratory gases, comprising: a first port and a second port and a fluid passageway formed therebetween; a valve arranged in the fluid passageway for controlling the flow of fluid between the first port and second port; a first sealing member having a first sealing surface arranged to seal against an interior surface of the fluid supply chamber proximate the first port when the valve unit is connected to the fluid supply chamber; a second sealing member having a second sealing surface arranged to seal against an exterior surface of the fluid supply chamber proximate the second port when the valve unit is connected to the fluid supply chamber.

In at least one example with reference to the first aspect, at least one of the first sealing member, the second sealing member, the valve, and/or the valve unit is reversibly or elastically deformable to put the valve unit under tension when connected to the fluid supply chamber.

In at least one example with reference to the first aspect, in a non-connected resting state, the first sealing surface and the second sealing surface are arranged at a closest distance from each other that is smaller than a closest distance between the first sealing surface and the second sealing surface when the valve unit is connected to the fluid supply chamber.

In at least one example with reference to the first aspect, the valve in a resting state is closed to prevent fluid entering the first port to flow therethrough towards the second port.

In at least one example with reference to the first aspect, the valve in an engaged state is open to allow fluid entering the first port to flow therethrough towards the second port.

In at least one example with reference to the first aspect, the valve attains its engaged state when a spike connector inserted into the fluid passageway engages the valve.

In at least one example with reference to the first aspect, the valve is a one-way valve or check valve.

In at least one example with reference to the first aspect, the valve is a duck bill valve arranged to open when a spike connector inserted into the fluid passageway engages the duck bill valve.

In at least one example with reference to the first aspect, the valve unit has a tubular or cylindrical cross-sectional shape.

In at least one example with reference to the first aspect, the first sealing member forms a flange and/or the second sealing member forms a flange.

In at least one example with reference to the first aspect, the first sealing member has a first material or structural characteristic, and the second sealing member has a second material or structural characteristic, wherein the first material or structural characteristic and the second material or structural characteristic are selected to allow for reversible deformation.

In at least one example with reference to the first aspect, the first material characteristic and second material characteristic relate to at least one of elastic modulus and yield stress.

In at least one example with reference to the first aspect, the first material characteristic and second material characteristic are the same.

In at least one example with reference to the first aspect, the first structural characteristic and/or second structural characteristic relate(s) to thickness.

In at least one example with reference to the first aspect, the valve unit is made of an elastomeric material.

In at least one example with reference to the first aspect, the first sealing surface is non-integrally formed with or detachable from the fluid supply chamber.

In at least one example with reference to the first aspect, the first sealing surface is integrally formed with the fluid supply chamber.

In at least one example with reference to the first aspect, the valve unit comprises a body extending between the first port and second port, wherein the body is made of a material that is reversibly or elastically deformable to allow the closest distance between the first sealing surface and the second sealing surface to increase from that of the non-connected state to that when the valve unit is connected to the fluid supply chamber, so that the valve unit is in tension when the valve unit is connected to the fluid supply chamber.

In at least one example with reference to the first aspect, the valve further comprises: a first flexible flap member having a first flap sealing surface extending between two respective lateral ends thereof, a second flexible flap member having a second sealing surface extending between two respective lateral ends thereof, wherein the first flap sealing surface and the second sealing surface are arranged to: engage each other to form a seal to close the valve in a resting state, and disengage from each other to open the valve in an engaged state to allow fluid to flow through the valve, wherein each lateral terminating end of the first flap sealing surface and the second flap sealing surface is coupled with an interior wall of the valve unit.

In at least one example with reference to the first aspect, each lateral terminating end of the first flap sealing surface is formed integral with a corresponding lateral terminating end of the second flap sealing surface.

In at least one example with reference to the first aspect, the first flap sealing surface and the second flap sealing surface in the engaged state engage at least part of an exterior surface the spike connector when the spike connector has been inserted into the fluid passageway to engage the valve.

In at least one example with reference to the first aspect, the interior wall forms part of the fluid passageway.

In at least one example with reference to the first aspect, the interior wall has a higher structural integrity than that of the first flexible flap member and/or second flexible flap member.

In at least one example with reference to the first aspect, the interior wall has a stiffness, thickness, and/or rigidity that is greater than that of the first flexible flap member and/or second flexible flap member.

In at least one example with reference to the first aspect, each lateral terminating end of the first flap sealing surface and the second flap sealing surface is formed integral with the interior wall of the valve unit.

In at least one example with reference to the first aspect, the fluid passageway is formed by an interior wall of the valve unit and extending along a longitudinal centre axis of the valve unit and, and the fluid passageway comprises: a first section of the interior wall proximal the second port having a first cross section, a second section of the interior wall extending along the longitudinal axis from an end of the first section, and having a second cross section that is larger than that of the first cross section, wherein the first section of the interior wall is arranged to engage a spike connector upon insertion of the spike connector into the fluid passageway to engage the valve.

In at least one example with reference to the first aspect, the valve is arranged between the first port and the second section.

In at least one example with reference to the first aspect, the second cross section is gradually increasing along the longitudinal axis towards the first port.

In at least one example with reference to the first aspect, the first section or second section extend symmetrically around the longitudinal centre axis.

In at least one example with reference to the first aspect, the first section is made of a material being reversibly or elastically deformable to put the first section under tension upon insertion of the spike connector into the fluid passageway to engage the valve.

In at least one example with reference to the first aspect, the first section forms a seal against a corresponding exterior section of the spike connector upon insertion of the spike connector into the fluid passageway to engage the valve.

In at least one example with reference to the first aspect, the first section forms an interference fit against an entire circumference of the spike connector upon insertion of the spike connector into the fluid passageway to engage the valve.

In at least one example with reference to the first aspect, the fluid passageway is formed along a longitudinal centre axis of the valve unit and wherein the valve is arranged in the fluid passageway longitudinally between the first port and second port for controlling the flow of fluid between the first port and second port.

In at least one example with reference to the first aspect, the valve is arranged in the fluid passageway along the longitudinal centre axis between a first point of the longitudinal centre axis and the second port, wherein the first point relates to a point of the longitudinal centre axis intersected by a plane comprising the first sealing surface.

In at least one example with reference to the first aspect, the valve is arranged in the fluid passageway along the longitudinal centre axis between a first point of the longitudinal centre axis and the second port, wherein the first point is defined by a point of the longitudinal axis intersected by a plane orthogonal to the longitudinal centre axis, which plane intersects a point of the first sealing surface that is closest to the second port.

In a second aspect there may be provided a valve unit for a fluid supply chamber of a respiratory support system that provides humidified respiratory gases, comprising: a first port and a second port and a fluid passageway formed therebetween; a valve arranged in the fluid passageway for controlling the flow of fluid between the first port and second port; the valve comprising a first flexible flap member having a first flap sealing surface extending between two respective lateral ends thereof, a second flexible flap member having a second sealing surface extending between two respective lateral ends thereof, wherein the first flap sealing surface and the second sealing surface are arranged to: engage each other to form a seal to close the valve in a resting state, and disengage from each other to open the valve in an engaged state to allow fluid to flow through the valve, wherein each lateral terminating end of the first flap sealing surface and the second flap sealing surface is coupled with an interior wall of the valve unit.

In at least one example with reference to the second aspect, each lateral terminating end of the first flap sealing surface is formed integral with a corresponding lateral terminating end of the second flap sealing surface.

In at least one example with reference to the second aspect, the first flap sealing surface and the second flap sealing surface in the engaged state engage at least part of an exterior surface the spike connector when the spike connector has been inserted into the fluid passageway to engage the valve.

In at least one example with reference to the second aspect, the interior wall forms part of the fluid passageway.

In at least one example with reference to the second aspect, the interior wall has a higher structural integrity than that of the first flexible flap member and/or second flexible flap member.

In at least one example with reference to the second aspect, the interior wall has a stiffness, thickness, and/or rigidity that is greater than that of the first flexible flap member and/or second flexible flap member.

In at least one example with reference to the second aspect, each lateral terminating end of the first flap sealing surface and the second flap sealing surface is formed integral with the interior wall of the valve unit.

In at least one example with reference to the second aspect, the valve unit comprises: a first sealing member having a first sealing surface arranged to seal against an interior surface of the fluid supply chamber proximate the first port when the valve unit is connected to the fluid supply chamber; a second sealing member having a second sealing surface arranged to seal against an exterior surface of the fluid supply chamber proximate the second port when the valve unit is connected to the fluid supply chamber.

In at least one example with reference to the second aspect, at least one of the first sealing member, the second sealing member, the valve, and/or the valve unit is reversibly or elastically deformable to put the valve unit under tension when connected to the fluid supply chamber.

In at least one example with reference to the second aspect, in a non-connected resting state, the first sealing surface and the second sealing surface are arranged at a closest distance from each other that is smaller than a closest distance between the first sealing surface and the second sealing surface when the valve unit is connected to the fluid supply chamber.

In at least one example with reference to the second aspect, the valve in a resting state is closed to prevent fluid entering the first port to flow therethrough towards the second port.

In at least one example with reference to the second aspect, the valve in an engaged state is open to allow fluid entering the first port to flow therethrough towards the second port.

In at least one example with reference to the second aspect, the valve attains its engaged state when a spike connector inserted into the fluid passageway engages the valve.

In at least one example with reference to the second aspect, the valve is a one-way valve or check valve.

In at least one example with reference to the second aspect, the valve is a duck bill valve arranged to open when a spike connector inserted into the fluid passageway engages the duck bill valve.

In at least one example with reference to the second aspect, the valve unit has a tubular or cylindrical cross-sectional shape.

In at least one example with reference to the second aspect, the first sealing member forms a flange and/or the second sealing member forms a flange.

In at least one example with reference to the second aspect, the first sealing member has a first material or structural characteristic, and the second sealing member has a second material or structural characteristic, wherein the first material or structural characteristic and the second material or structural characteristic are selected to allow for reversible deformation.

In at least one example with reference to the second aspect, the first material characteristic and second material characteristic relate to at least one of elastic modulus and yields stress.

In at least one example with reference to the second aspect, the first material characteristic and second material characteristic are the same.

In at least one example with reference to the second aspect, the first structural characteristic and/or second structural characteristic relate(s) to thickness.

In at least one example with reference to the second aspect, the valve unit is made of an elastomeric material.

In at least one example with reference to the second aspect, the first sealing surface is non-integrally formed with or detachable from the fluid supply chamber.

In at least one example with reference to the second aspect, the first sealing surface is integrally formed with the fluid supply chamber.

In at least one example with reference to the second aspect, the valve unit further comprises a body extending between the first port and second port, wherein the body is made of a material that is reversibly or elastically deformable to allow the closest distance between the first sealing surface and the second sealing surface to increase from that of the non-connected state to that when the valve unit is connected to the fluid supply chamber, so that the valve unit is in tension when the valve unit is connected to the fluid supply chamber.

In at least one example with reference to the second aspect, the body comprises the interior wall.

In at least one example with reference to the second aspect, the fluid passageway is formed by the interior wall and extending along a longitudinal centre axis of the valve unit and, the fluid passageway comprising: a first section of the interior wall proximal the second port having a first cross section, a second section of the interior wall extending along the longitudinal axis from an end of the first section, and having a second cross section that is larger than that of the first cross section, wherein the first section of the interior wall is arranged to engage a spike connector upon insertion of the spike connector into the fluid passageway to engage the valve.

In at least one example with reference to the second aspect, the valve is arranged between the first port and the second section.

In at least one example with reference to the second aspect, the second cross section is gradually increasing along the longitudinal axis towards the first port.

In at least one example with reference to the second aspect, the first section or second section extend symmetrically around the longitudinal centre axis.

In at least one example with reference to the second aspect, the first section is made of a material being reversibly or elastically deformable to put the first section under tension upon insertion of the spike connector into the fluid passageway to engage the valve.

In at least one example with reference to the second aspect, the first section forms a seal against a corresponding exterior section of the spike connector upon insertion of the spike connector into the fluid passageway to engage the valve.

In at least one example with reference to the second aspect, the first section forms an interference fit against an entire circumference of the spike connector upon insertion of the spike connector into the fluid passageway to engage the valve.

In at least one example with reference to the second aspect, the fluid passageway is formed along a longitudinal centre axis of the valve unit and wherein the valve is arranged in the fluid passageway longitudinally between the first port and second port for controlling the flow of fluid between the first port and second port.

In at least one example with reference to the second aspect, the valve is arranged in the fluid passageway along the longitudinal centre axis between a first point of the longitudinal centre axis and the second port, wherein the first point relates to a point of the longitudinal centre axis intersected by a plane comprising the first sealing surface.

In at least one example with reference to the second aspect, the valve is arranged in the fluid passageway along the longitudinal centre axis between a first point of the longitudinal centre axis and the second port, wherein the first point is defined by a point of the longitudinal axis intersected by a plane orthogonal to the longitudinal centre axis, which plane intersects a point of the first sealing surface that is closest to the second port.

In a third aspect there may be provided a valve unit for a fluid supply chamber of a respiratory support system that provides humidified respiratory gases, comprising: a first port and a second port and a fluid passageway formed therebetween; a valve arranged in the fluid passageway for controlling the flow of fluid between the first port and second port; wherein the fluid passageway is formed by an interior wall of the valve unit and extending along a longitudinal centre axis of the valve unit and, and the fluid passageway comprising: a first section of the interior wall proximal the second port having a first cross section, a second section of the interior wall extending along the longitudinal axis from an end of the first section, and having a second cross section that is larger than that of the first cross section, wherein the first section of the interior wall is arranged to engage a spike connector upon insertion of the spike connector into the fluid passageway to engage the valve.

In at least one example with reference to the third aspect, the valve is arranged between the first port and the second section.

In at least one example with reference to the third aspect, the second cross section is gradually increasing along the longitudinal axis towards the first port.

In at least one example with reference to the third aspect, the first section or second section extend symmetrically around the longitudinal centre axis.

In at least one example with reference to the third aspect, the first section is made of a material being reversibly or elastically deformable to put the first section under tension upon insertion of the spike connector into the fluid passageway to engage the valve.

In at least one example with reference to the third aspect, the first section forms a seal against a corresponding exterior section of the spike connector upon insertion of the spike connector into the fluid passageway to engage the valve.

In at least one example with reference to the third aspect, the first section forms an interference fit against an entire circumference of the spike connector upon insertion of the spike connector into the fluid passageway to engage the valve.

In at least one example with reference to the third aspect, the valve unit comprises: a first sealing member having a first sealing surface arranged to seal against an interior surface of the fluid supply chamber proximate the first port when the valve unit is connected to the fluid supply chamber; a second sealing member having a second sealing surface arranged to seal against an exterior surface of the fluid supply chamber proximate the second port when the valve unit is connected to the fluid supply chamber.

In at least one example with reference to the third aspect, at least one of the first sealing member, the second sealing member, the valve, and/or the valve unit is reversibly or elastically deformable to put the valve unit under tension when connected to the fluid supply chamber.

In at least one example with reference to the third aspect, in a non-connected resting state, the first sealing surface and the second sealing surface are arranged at a closest distance from each other that is smaller than a closest distance between the first sealing surface and the second sealing surface when the valve unit is connected to the fluid supply chamber.

In at least one example with reference to the third aspect, the valve in a resting state is closed to prevent fluid entering the first port to flow therethrough towards the second port.

In at least one example with reference to the third aspect, the valve in an engaged state is open to allow fluid entering the first port to flow therethrough towards the second port.

In at least one example with reference to the third aspect, the valve attains its engaged state when a spike connector inserted into the fluid passageway engages the valve.

In at least one example with reference to the third aspect, the valve is a one-way valve or check valve.

In at least one example with reference to the third aspect, the valve is a duck bill valve arranged to open when a spike connector inserted into the fluid passageway engages the duck bill valve.

In at least one example with reference to the third aspect, the valve unit has a tubular or cylindrical cross-sectional shape.

In at least one example with reference to the third aspect, the first sealing member forms a flange and/or the second sealing member forms a flange.

In at least one example with reference to the third aspect, the first sealing member has a first material or structural characteristic, and the second sealing member has a second material or structural characteristic, wherein the first material or structural characteristic and the second material or structural characteristic are selected to allow for reversible deformation.

In at least one example with reference to the third aspect, the first material characteristic and second material characteristic relate to at least one of elastic modulus and yields stress.

In at least one example with reference to the third aspect, the first material characteristic and second material characteristic are the same.

In at least one example with reference to the third aspect, the first structural characteristic and/or second structural characteristic relate(s) to thickness.

In at least one example with reference to the third aspect, the valve unit is made of an elastomeric material.

In at least one example with reference to the third aspect, the first sealing surface is non-integrally formed with or detachable from the fluid supply chamber.

In at least one example with reference to the third aspect, the first sealing surface is integrally formed with the fluid supply chamber.

In at least one example with reference to the third aspect, the valve unit further comprises a body extending between the first port and second port, wherein the body is made of a material that is reversibly or elastically deformable to allow the closest distance between the first sealing surface and the second sealing surface to increase from that of the non-connected state to that when the valve unit is connected to the fluid supply chamber, so that the valve unit is in tension when the valve unit is connected to the fluid supply chamber.

In at least one example with reference to the third aspect, the body comprises the interior wall.

In at least one example with reference to the third aspect, the valve further comprises: a first flexible flap member having a first flap sealing surface extending between two respective lateral ends thereof, a second flexible flap member having a second sealing surface extending between two respective lateral ends thereof, wherein the first flap sealing surface and the second sealing surface are arranged to: engage each other to form a seal to close the valve in a resting state, and disengage from each other to open the valve in an engaged state to allow fluid to flow through the valve, wherein each lateral terminating end of the first flap sealing surface and the second flap sealing surface is coupled with the interior wall of the valve unit.

In at least one example with reference to the third aspect, each lateral terminating end of the first flap sealing surface is formed integral with a corresponding lateral terminating end of the second flap sealing surface.

In at least one example with reference to the third aspect, the first flap sealing surface and the second flap sealing surface in the engaged state engage at least part of an exterior surface the spike connector when the spike connector has been inserted into the fluid passageway to engage the valve.

In at least one example with reference to the third aspect, the interior wall forms part of the fluid passageway.

In at least one example with reference to the third aspect, the interior wall has a higher structural integrity than that of the first flexible flap member and/or second flexible flap member.

In at least one example with reference to the third aspect, the interior wall has a stiffness, thickness, and/or rigidity that is greater than that of the first flexible flap member and/or second flexible flap member.

In at least one example with reference to the third aspect, each lateral terminating end of the first flap sealing surface and the second flap sealing surface is formed integral with the interior wall of the valve unit.

In at least one example with reference to the third aspect, the fluid passageway is formed along a longitudinal centre axis of the valve unit and wherein the valve is arranged in the fluid passageway longitudinally between the first port and second port for controlling the flow of fluid between the first port and second port.

In at least one example with reference to the third aspect, the valve is arranged in the fluid passageway along the longitudinal centre axis between a first point of the longitudinal centre axis and the second port, wherein the first point relates to a point of the longitudinal centre axis intersected by a plane comprising the first sealing surface.

In at least one example with reference to the third aspect, the valve is arranged in the fluid passageway along the longitudinal centre axis between a first point of the longitudinal centre axis and the second port, wherein the first point is defined by a point of the longitudinal axis intersected by a plane orthogonal to the longitudinal centre axis, which plane intersects a point of the first sealing surface that is closest to the second port.

In a fourth aspect there may be provided an assembly for a respiratory support system that provides humidified respiratory gases, comprising: a valve unit as disclosed herein, and at least one of the following: a fluid supply chamber for connection to the valve unit; a spike connector; a supply tube at a first end thereof arranged to connect to the spike connector; a humidification chamber for connection to a second end of the supply tube; a humidifier comprising the humidification chamber for connection to the second end of the supply tube; a gases source; a patient interface for connection to the gases source or humidifier by a gases supply tube; and a gases supply tube for connection between the gases source and/or humidifier and/or humidification chamber and the patient interface.

In at least one example with reference to the fourth aspect, the humification chamber comprises an inlet for receiving fluid from the supply tube, and one or more floats for maintaining a fluid level in the humidification chamber when the supply tube holding fluid is connected to the inlet.

In at least one example with reference to the fourth aspect, the supply tube is releasably or permanently attached to an inlet of the humidification chamber.

In at least one example with reference to the fourth aspect, the spike connector is releasably or permanently attached to the first end of the supply tube.

In a fifth aspect there may be provided a fluid supply chamber assembly for a respiratory support system that provides humidified respiratory gases, comprising a valve unit as disclosed herein, and a fluid supply chamber arranged to hold a volume of liquid therein and having a fluid access port, wherein the valve unit is connected to the fluid access port.

### BRIEF DESCRIPTION OF THE DRAWINGS

A number of embodiments will now be shown, by way of example, with reference to the following drawings, in which:

FIG. 1 illustrates a respiratory assistance system of the prior art;

FIGS. 2 to 7 illustrate respective perspective views of a valve unit according to an embodiment;

FIGS. 8 to 11 illustrate respective cross-sectional views of the valve unit of FIGS. 2 to 7;

FIGS. 12a and 12b illustrate two respective different states of the valve unit of FIGS. 2 to 11;

FIGS. 14 to 15 illustrate respective perspective views of a fluid supply chamber according to an embodiment;

FIG. 16 illustrates a cross-sectional view of the fluid supply chamber of FIGS. 14 to 15;

FIG. 17 illustrates a cross-sectional view of a fluid access port of the fluid supply chamber, for receiving the valve unit according to an embodiment;

FIG. 18 illustrates a rear perspective view of a fluid supply chamber assembly comprising the fluid supply chamber of FIGS. 14 to 17 and the valve unit of FIGS. 2 to 12b connected to the fluid access port;

FIG. 19 illustrates an enlarged portion of FIG. 18 showing the attached valve unit in its connected state;

FIG. 20 illustrates a cross sectional view of the fluid supply chamber assembly of FIGS. 18 to 19;

FIG. 21 illustrates an enlarged portion of FIG. 20 showing the valve unit at an intermediate stage upon connection to the fluid access port of the fluid supply chamber;

FIG. 22 illustrates an enlarged portion of FIG. 20 showing the valve unit in its connected state in the fluid access port of the fluid supply chamber;

FIGS. 23 and 24 illustrate respective cross-sectional views of the fluid supply chamber assembly according to an embodiment;

FIG. 25 illustrates a perspective view of the fluid supply chamber assembly showing the valve unit in its connected and/or installed state in the fluid access port of the fluid supply chamber;

FIG. 26 illustrates a cross-sectional view of the fluid supply chamber assembly upon receiving a spike connector according to an embodiment;

FIGS. 27 and 28 illustrate respective enlarged views of FIG. 26 showing the valve unit when connected and/or installed to the fluid supply chamber and the spike connector positioned for insertion into the valve unit;

FIG. 29 illustrates a cross-sectional view of the fluid supply chamber assembly when the spike connector has been fully inserted into the valve unit;

FIG. 30 illustrates an enlarged view of FIG. 29;

FIGS. 31 and 32 illustrates respective perspective views of the valve unit when the spike connector has reached its fully inserted state according to an embodiment; and

FIG. 33 illustrates an assembly according to an embodiment.

### DETAILED DESCRIPTION

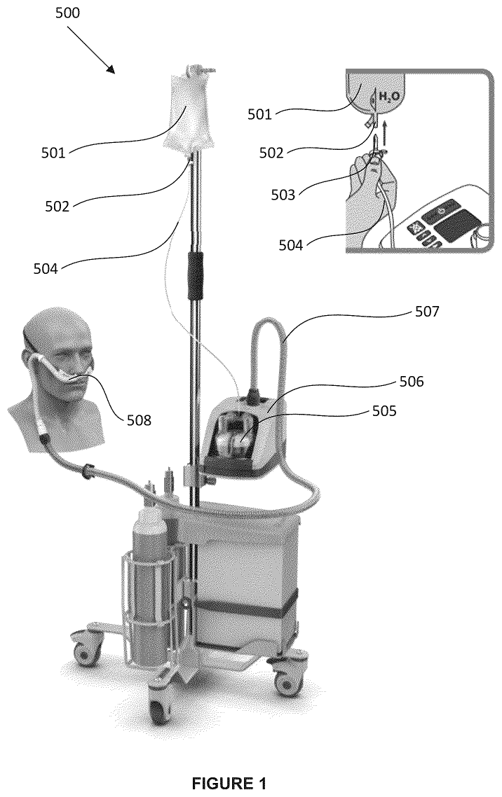

FIG. 1 illustrates an example respiratory support system 500 comprising an auxiliary fluid supply chamber 501 connected to a humidification chamber 505. The humidification chamber 505 contains a fluid, such as water, used to humidify the gases flow flowing through the humidification chamber 505. The humidification chamber 505 is connected to a gases source 506, e.g. a flow generator. Humidified air leaving the humidification chamber 505 is delivered to a patient interface 508 via a gas supply tube 507. Sometimes the volume of water that the humidification chamber 505 can hold is not sufficient for the entire therapy session, whereby the water may run out before the therapy session ends. This issue can be addressed by connecting an auxiliary fluid supply chamber 501, such as a water bottle, to the humidification chamber 505. The humidification chamber 505 may comprise a float system with one or more floats that respond to the level of fluid within the humidification chamber 505. The float system may include a valve at a water inlet of the humidification chamber. The valve may be controlled based on the float position. The valve opens when the water level is below a threshold level and the valve closes when the water level is above the threshold level. The relative position of said float(s) within the humidification chamber will dictate when fluid, e.g. water, from the auxiliary fluid supply chamber is able to flow into the humidification chamber. Such a float system can maintain a consistent volume of fluid in the humidification chamber provided there is fluid present in the auxiliary fluid supply chamber.

Auxiliary fluid supply chambers are typically not permanently attached to humidification chambers, because, for example, the user may need to separate the auxiliary chamber from the respiratory device to refill it. As shown with reference to FIG. 1, the humidification chamber 505 has a permanently attached tube 504 that has a spike connector 503 on the other end. To access the fluid of the auxiliary fluid supply chamber this spike connector 503 needs to be inserted into an off the shelf spike port 502 that is connected to the auxiliary fluid supply chamber 501, as shown with reference to FIG. 1. To insert the spike connector 503, the user, such as the patient, needs to pierce a membrane on the spike port 502 with the tip of the spike connector 503. However, as many users of respiratory devices are physically compromised, it may prove difficult to correctly pierce the spike port membrane, which in turn may lead to damage to the spike port, and undesired leakage.

The present disclosure relates to a valve unit (hereinafter also referred to as a “bottle spike port”) and associated fluid supply chamber. The valve unit configured to enable fluid to flow from the fluid supply chamber to a humidification chamber of a respiratory support system

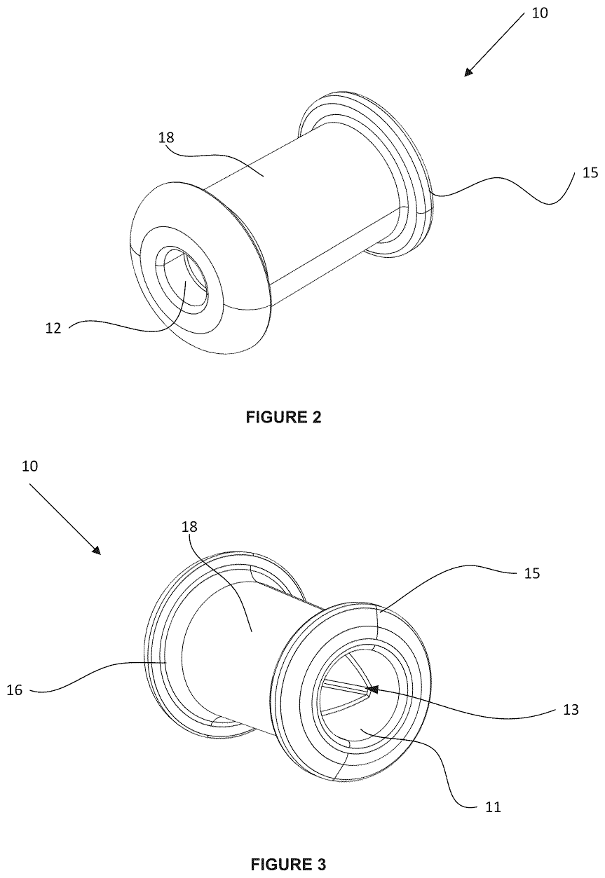

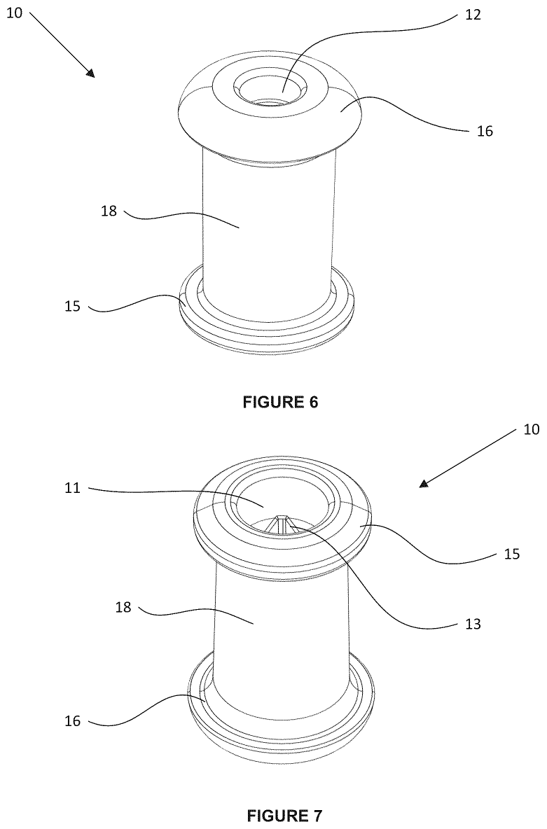

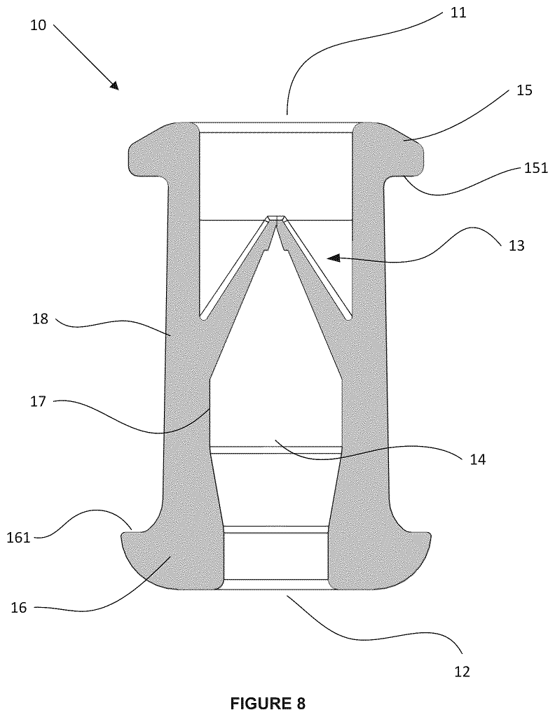

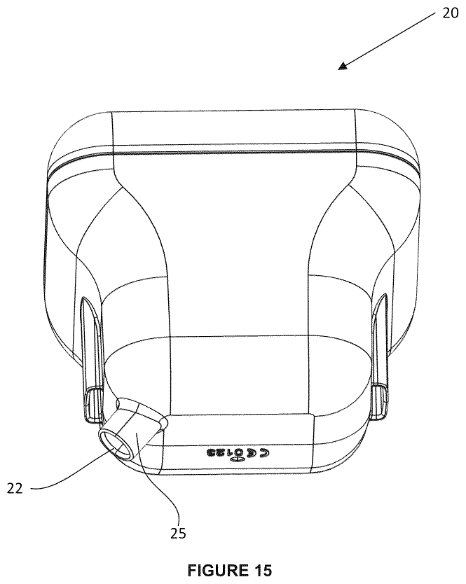

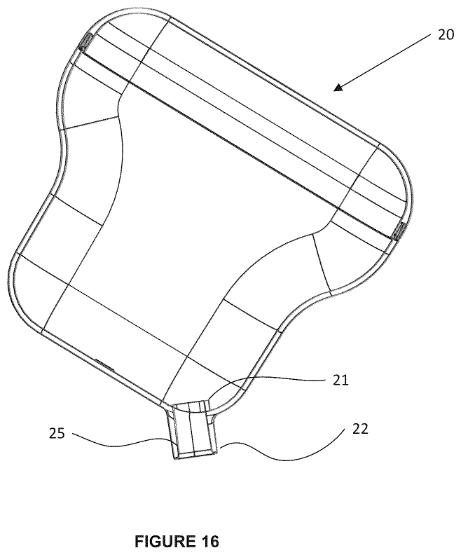

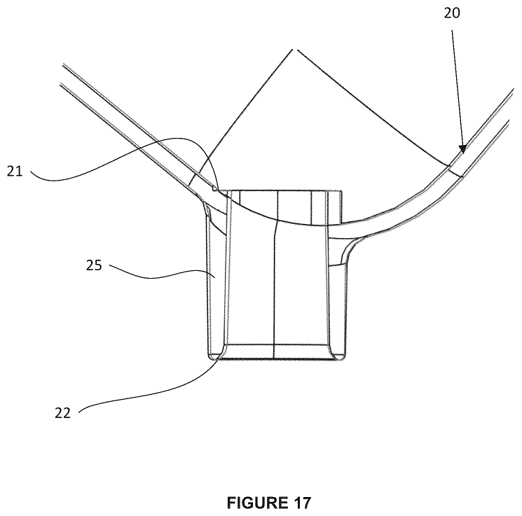

FIGS. 2 to 12b illustrate various views of a valve unit 10 according to a first embodiment. The valve unit 10 is arranged to connect to a fluid supply chamber 20 of a respiratory support system, such as that shown with reference to FIGS. 13 to 17.

The valve unit 10 has an elongate body 18 and comprises a first port 11 and a second port 12 formed within the body 18. The first port 11 and the second port 12 are configured to allow fluid entry into the elongate body 18.

A fluid passageway 14 is formed between the first port 11 and second port 12, such that fluid is able to flow into and out of the first port 11 and/or the second port 12, and between the second port 12 and the first port 11.

The valve unit 10 further comprises a valve 13 arranged in the fluid passageway 14 for controlling the flow of fluid between the first port 11 and second port 12.

Fluid may flow through the valve when the valve is open, for example via a spike connector engaging the valve in an engaged state as will be further elucidated below and prevent from such flow when the valve is closed or in a disengaged state.

The valve unit 10 comprises a first sealing member 15 proximate the first port 11 that has a first sealing surface 151. The valve unit further comprises a second sealing member 16 proximate the second port that has a second sealing surface 161. The first sealing member 15 and the second sealing member 16 may be arranged such that the first sealing surface 151 and the second sealing surface 161 are substantially facing towards each other.

The first sealing member 15 and/or the second sealing member 16 may each form a flange which is adapted for sealing against a surface of the fluid supply chamber 20. The first sealing member 15 and/or the second sealing member 16 may be a circumferential flange around the first port 11 and the second port 12, respectively.

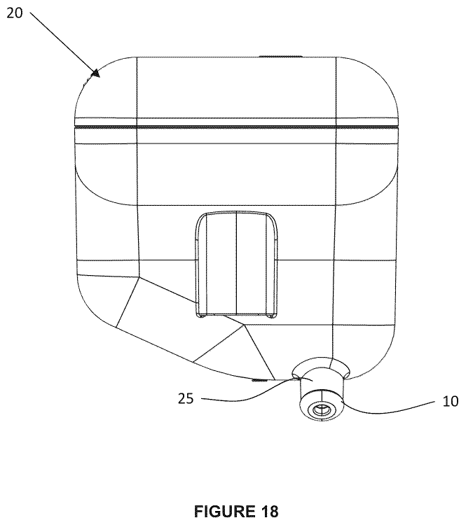

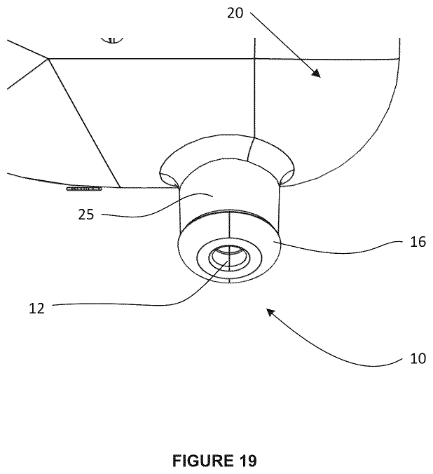

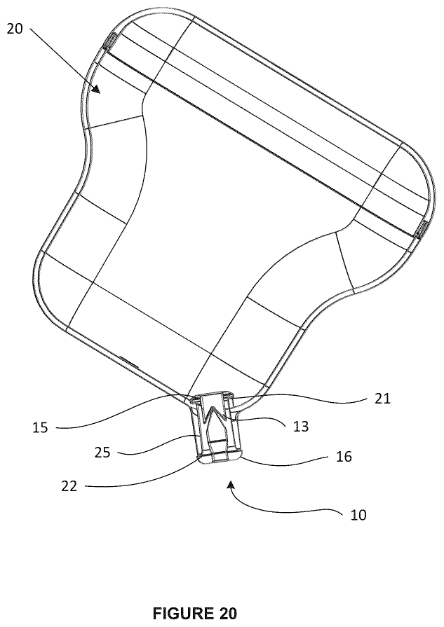

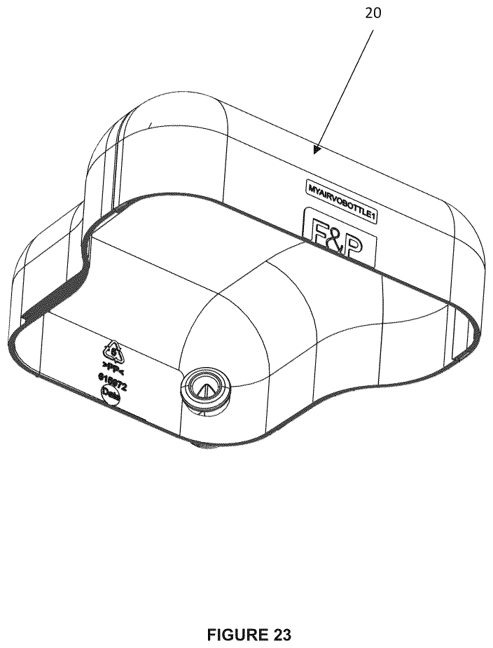

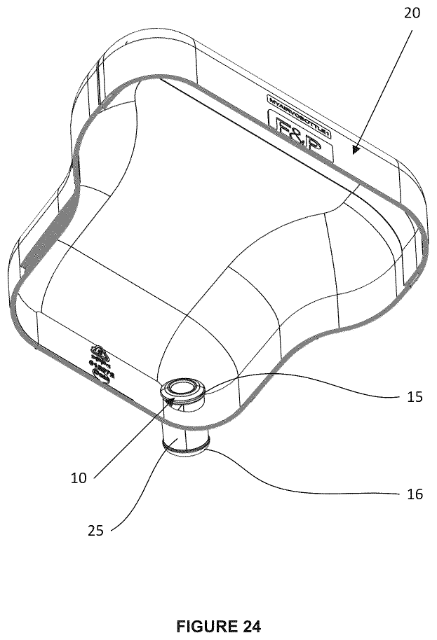

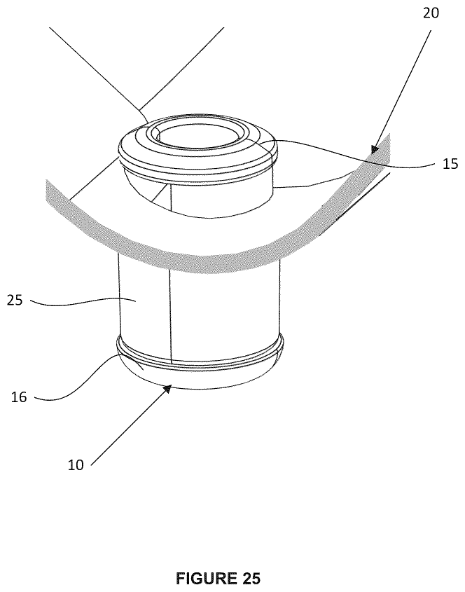

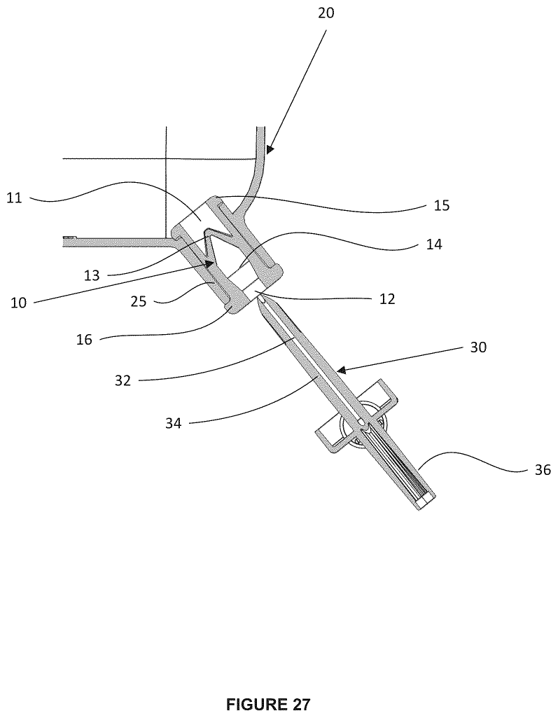

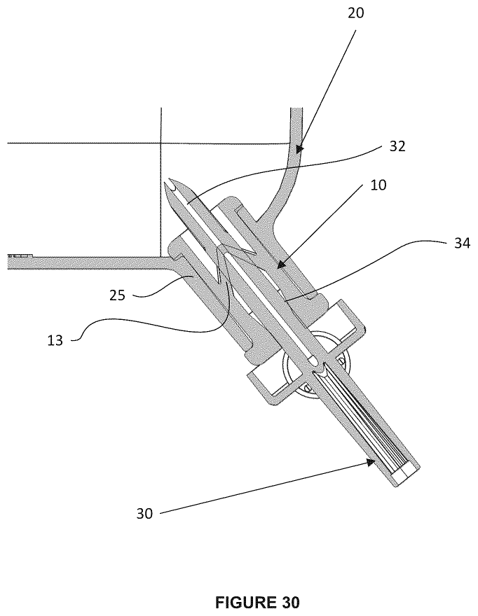

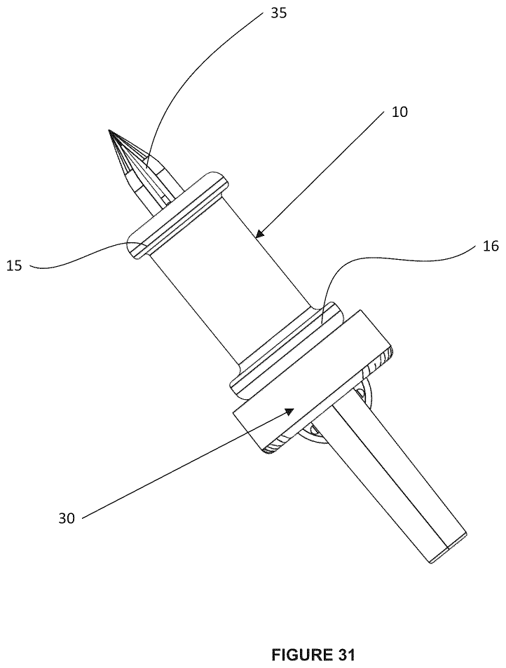

FIGS. 18 to 30 illustrate various views of the valve unit 10 of FIGS. 2 to 12b connected to the fluid supply chamber 20 of FIGS. 13 to 17.

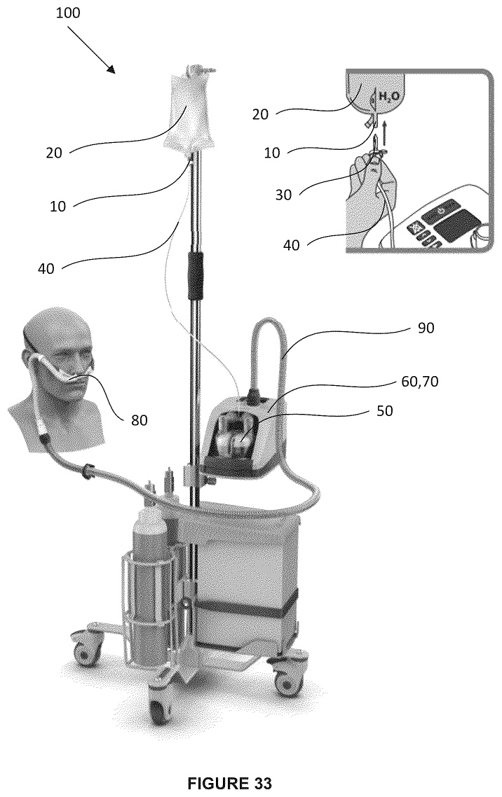

The fluid supply chamber may comprise a solid or rigid body, e.g. formed from a solid plastics material. Alternatively, the fluid supply chamber may comprise a flexible body, such as a flexible bag, as shows with reference to FIG. 33. The valve unit disclosed herein may be incorporated into either type of fluid supply chamber. The first sealing member 15, more specifically a first sealing surface 151 of the first sealing member 15, is arranged to seal against an interior surface 21 of the fluid supply chamber 20 when the valve unit 10 is connected to the fluid supply chamber 20.

The second sealing member 16, more specifically a second sealing surface 161 of the second sealing member 16, is arranged to seal against an exterior surface 22 of the fluid supply chamber 20 proximate the second port 12 when the valve unit 10 is connected to the fluid supply chamber 20.

At least one of the first sealing member 15, the second sealing member 16, the valve 13, and/or the elongate body 18 of the valve unit 10 is reversibly or elastically deformable.

When the valve unit 10 is connected to the fluid supply chamber 20, the first sealing member 15 seals against an interior surface 21 of the fluid supply chamber 20, and the second sealing member 16 seals against an exterior surface 22 of the fluid supply chamber 20.

The reversible or elastic deformation of at least one of the first sealing member 15, the second sealing member 16, the valve 13, and/or the elongate body 18 of the valve unit 10, allows for the valve unit 10, when connected to the fluid supply chamber 20, to be held in a state of tension along its elongate length or at least a substantial portion of its elongate length.

This state of tension along the elongate length or at least a substantial portion of the elongate length of the valve unit 10 results in a watertight or substantially watertight seal between at least the first sealing member 15 and the interior surface 21 of the fluid supply chamber 20.

Furthermore, the sealing between the first sealing member 15 and the interior surface 21 of the fluid supply chamber 20, when the valve unit 10 is connected to the fluid supply chamber 20, means that the valve unit 10 is tightly held within the fluid supply chamber 20. To remove or disconnect the valve unit 10 from the fluid supply chamber 20 a considerable amount of pulling force along the length of the valve unit is required. Thus, once the valve unit is connected to the fluid supply chamber it is not easily disconnected and this reduces the risk of accidental removal of the valve unit 10 from the fluid supply chamber 20.

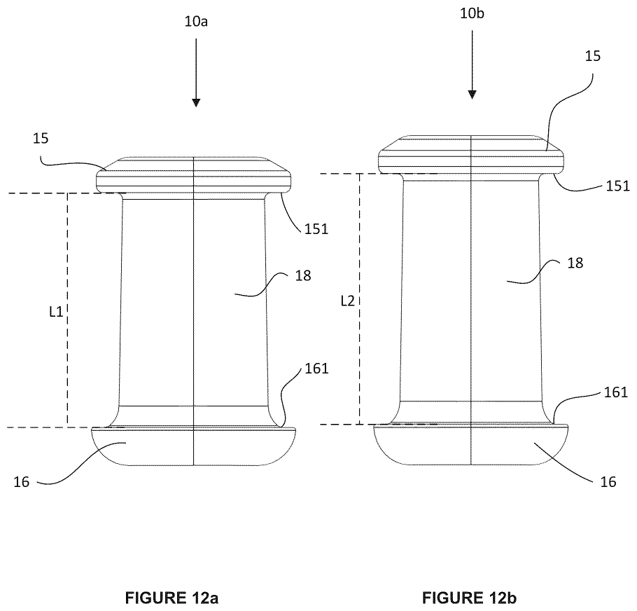

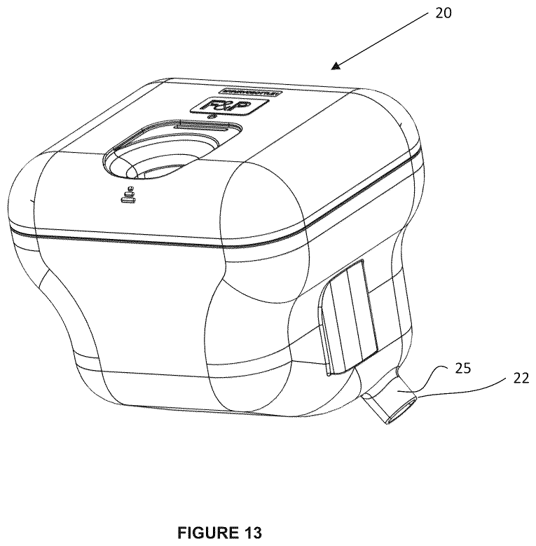

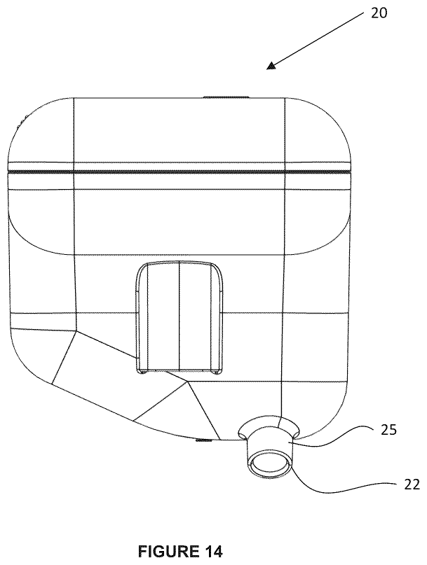

FIGS. 12a and 12b illustrate two respective different states of the valve unit of FIGS. 2 to 11.

As shown in FIG. 12a, the valve unit 10 is in a non-connected resting state. In this non-connected resting state, the first sealing surface 151 and the second sealing surface 161 are arranged at a distance L1 from each other.

When the valve unit 10 is connected to the fluid supply chamber 20 as described above, the valve unit 10 is held in a state of tension. As shown in FIG. 12b, the valve unit 10 is in a connected state. In this connected state, the first sealing surface 151 and the second sealing surface 161 are arranged at a distance L2 from each other.

As shown, the distance L1 when the valve unit 10 is in the non-connected resting state is smaller than the distance L2 between the first sealing surface 151 and the second sealing surface 161 when the valve unit 10 is connected to the fluid supply chamber 20, in the connected state.

The increased distance between the first sealing surface 151 and the second sealing surface 161 when in the connected state is by way of the reversible or elastic deformation of at least one of the first sealing member 15, the second sealing member 16, the valve 13, and/or the elongate body 18 of the valve unit 10.

The distance L2 when in the connected state is greater than the distance L1 when the valve unit 10 is in a non-connected resting state. The difference between L1 and L2 may be between about 0.1 mm and about 10 mm. More preferably, the difference between L1 and L2 is between about 0.1 mm and 1.0 mm, and more preferably is about 0.5 mm.

In the embodiments shown, a valve 13 is arranged in the fluid passageway 14 for controlling the flow of fluid between the first port 11 and second port 12.

The valve 13 may be a one-way valve or check valve according to some embodiments. In such embodiments, the valve 13 is arranged to allow fluid under pressure to flow through it in only one direction when the valve 13 is in a disengaged state. Further the valve 13 may be arranged to allow fluid to flow though it in both directions when the valve 13 is in an engaged state (as will be further elucidated below).

For example, in some embodiments, in a disengaged state fluid under pressure at the second port 12 could be used to force open the valve to allow the pressurised fluid to flow between the second port 12 and the first port 11 for example to allow for refilling of the fluid supply chamber with fluid. The pressure required to force open the valve may need to meet a threshold pressure. Alternatively, or additionally a separate port may be provided to the fluid supply chamber to allow for refilling of the fluid supply chamber.

Further, in the disengaged state fluid is prevented from flowing from the first port 11 to the second port 12. Such a configuration allows for preventing undesired release of fluid from the fluid supply chamber when the valve unit is connected thereto and is in its disengaged state as the fluid is prevented from passing through the valve 13 in a direction from the first port 11 towards the second port 12.

As described above, the valve unit 10 is configured to be connected to the fluid supply chamber 20 whereby the first sealing member 15 is arranged to seal against an interior surface 21 of the fluid supply chamber 20 proximate the first port 11, and the second sealing member 16 is arranged to seal against an exterior surface 22 of the fluid supply chamber 20 proximate the second port 12 when the valve unit 10 is connected to the fluid supply chamber 20.

A such, when the valve unit 10 is connected to the fluid supply chamber 20, the valve 13 is arranged in the fluid passageway 14 between the first port 11 and the second port 12 and is positioned such that fluid is prevented from flowing from the first port 11 to the second port 12 when the valve 13 is in its disengaged state.

In further such embodiments, the valve 13 may be a check valve or one-way valve in the form of a duck bill valve. The duck bill valve is arranged such that it prevents fluid from flowing between the first port 11 to the second port 12 when in a closed position relating to the disengaged state.

The valve 13 is arranged to open when a spike connector is inserted into the fluid passageway and 14 engages the valve 13. Hence in the engaged state the valve 13 is open and allows for fluid to flow through the valve 13 via the hollow spike connector.

As the valve unit 10 is typically arranged at the lowermost end of the fluid supply chamber 20, when in its engaged state fluid from the fluid supply chamber 20 is allowed to flow through the valve between the first port 11 and second port 12 under the influence of gravity. Hence, in some embodiments, gravitational forces may thus be said to influence the direction of travel of the fluid through the valve unit when the valve unit is in its engaged state.

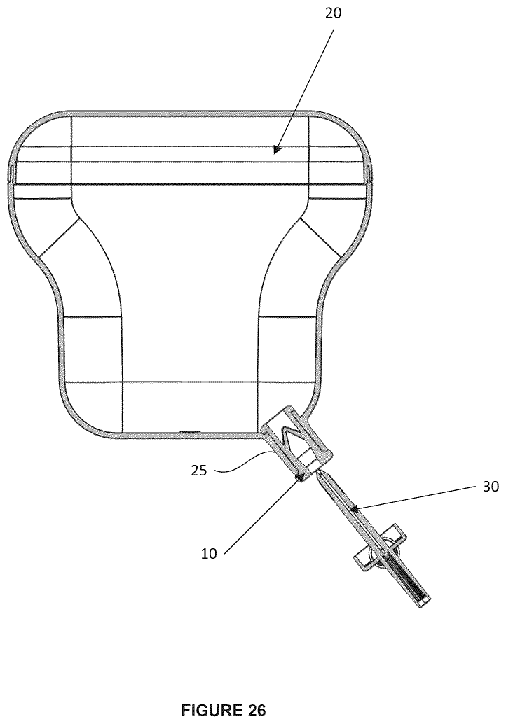

As shown with reference to FIGS. 26 to 28, when the valve 13 is not engaged with the spike connector 30, the valve unit 10 is in a disengaged state. In the disengaged state, the valve 13 is closed and prevents any fluid entering the first port 11 to flow therethrough towards the second port 12 through the fluid passageway 14.

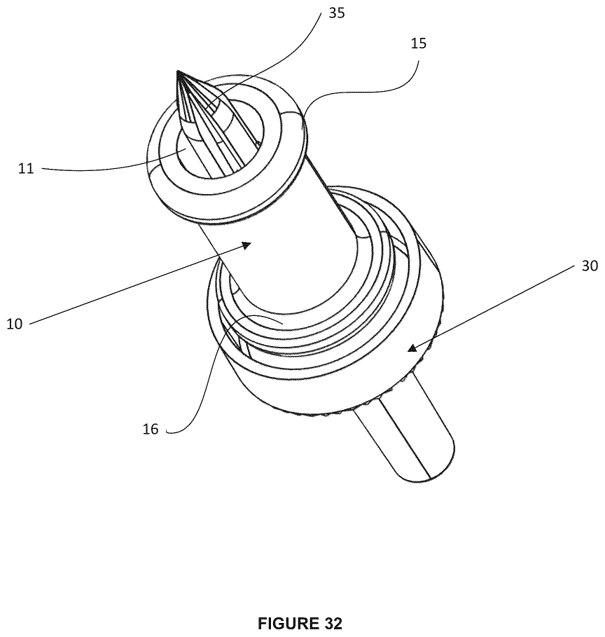

As shown with reference to FIGS. 29 to 32, the valve 13 attains its engaged state when the spike connector 30 is inserted into second port 12, through the fluid passageway 14. The body 34 of the spike connector 30 thereby engages the valve 13. When the valve 13 is engaged with the body 34 of the spike connector 30 and the valve unit 10 is in the engaged state, the valve 13 is open and allows fluid entering the first port 11 to flow therethrough towards the second port 12. Fluid from the fluid supply chamber 20 may flow into the fluid passageway 32 of the spike connector and to a conduit which may be attached to the distal end 36 of the spike connector 30.

As discussed above, the valve unit 10 is arranged to connect to a fluid supply chamber 20 of a respiratory support system, such as that shown with reference to FIGS. 13 to 17. The valve unit is configured to be inserted into, and to be held within, a fluid access port 25 of the fluid supply chamber. The fluid access port 25 may form an outlet/inlet of the fluid supply chamber 20.

The fluid access port 25 may have an interior shape conforming with an exterior shape of the valve unit 10 to form a seal, interference fit, or friction fit, with the valve unit 10 when connected to the fluid supply chamber.

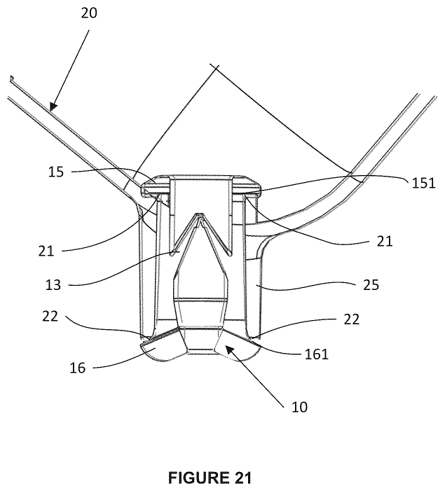

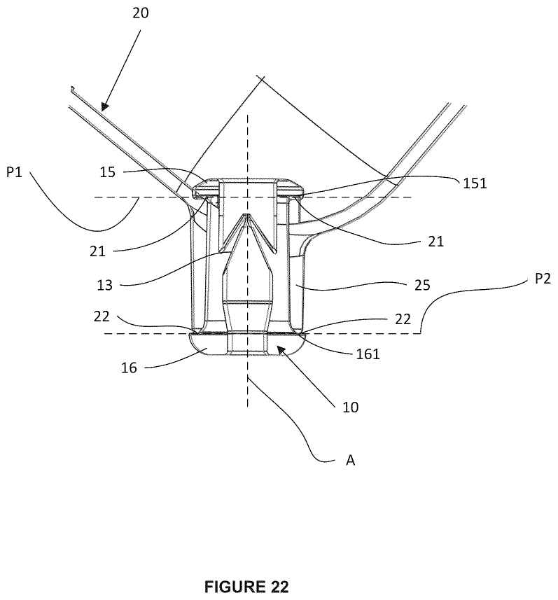

FIG. 21 illustrates an enlarged portion of FIG. 20 showing the valve unit at an intermediate stage upon connection to the fluid access port of the fluid supply chamber. At this intermediate stage the valve unit has been inserted into the fluid access port 25 and the first sealing surface 151 has just engaged the interior surface 21 of the fluid supply chamber 20. The other end of the valve unit comprising the second port 12 and the second sealing surface 161 is somewhat pressed into the fluid access chamber 20. The second port 12 could be pressed into the fluid access port 25 for example by pushing the valve unit into the fluid access port 25 by means of a finger or other suitable tool to allow the first sealing member 15 to exit the fluid access port 25 inside the fluid supply chamber to allow the first sealing surface 151 to engage the interior surface 21 thereof. In the depressed state the second sealing surface 161 engage the exterior surface 22 of the fluid supply chamber while the second sealing member is deflected from its resting state configuration. Once the first sealing surface 151 has engaged the interior surface 21 of the fluid supply chamber the valve unit may be released whereby it returns to configuration as shown in FIG. 22. Upon releasing the valve unit 10, i.e. upon removal of the load or force exerted on the valve unit during insertion into the fluid access port 25, the closest distance between the first sealing member 151 and the second sealing member 161 increases from L1 to L2 as shown with reference to FIGS. 12a and 12b. This puts the valve unit in tension, and the tension acts to pull the first sealing surface 151 towards the second sealing surface 161, whereby the first sealing surface 151 is forced against the interior surface 21 and the second sealing surface 161 is forced against the exterior surface 22, thereby forming a seal.

In some embodiments, the first sealing surface 151 is non-integrally formed with or detachable from the fluid supply chamber 20. In alternative embodiments, the first sealing surface 151 is integrally formed with the fluid supply chamber 20.

The shape of the valve unit 10 is configured to substantially correspond to the shape of the fluid access port 25 of the fluid supply chamber 20. In the embodiment shown, the elongate body 18 of the valve unit 10 has a circular, tubular or cylindrical cross-sectional shape. The tubular or cylindrical cross-sectional shape of the elongate body 18 corresponds to the tubular or cylindrical cross-sectional shape of the fluid access port 25 of the fluid supply chamber 20. In alternative embodiments, the elongate body 18 of the valve unit 10 may have a different cross-sectional shape, and may take the form of a square, or another regular polygon or any other shape adapted to fit in the fluid access port.

The substantial correspondence of the shape of the valve unit 10 to the shape of the fluid access port 25 of the fluid supply chamber 20 may provide for a seal between the exterior of the elongate body 18 of the valve unit 10 and the internal wall of the fluid access port 25 of the fluid supply chamber 20. The seal may be watertight such that fluid from the fluid supply chamber is prevented from exiting the fluid supply chamber by way of the contact between the exterior of the elongate body 18 of the valve unit 10 and the internal wall of the fluid access port 25 of the fluid supply chamber 20.

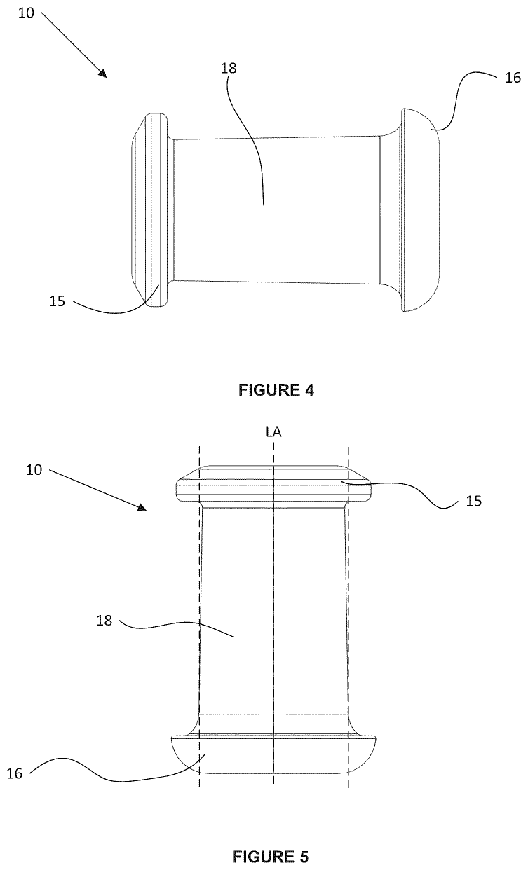

The portion of the elongate body 18 between the first sealing surface 151 and the second sealing surface 161 may have a variable cross-sectional shape. For example, as shown in the side views of a valve unit 10 of FIGS. 4 and 5, the cross-sectional shape of the portion of the elongate body 18 is greater towards the second sealing member 16 and lesser towards the first sealing member 15.

According to some embodiments, the variable cross-sectional shape is tapered from the second port 12 towards the first port 11. As may be observed from FIG. 5 the cross-sectional shape of the body 18 of the valve unit 10 is tapered with reference to the two dashed lines each being parallel to the longitudinal axis LA of the valve unit.

The varied cross-sectional shape shown in this embodiment may allow for a facilitated insertion of the valve unit 10 into the fluid supply chamber 20 upon connection. In alternate embodiments, the cross-sectional shape of the portion of the elongate body 18 between the first sealing surface 151 and the second sealing surface 161 may be uniform.

The body 18 of the valve unit 10 is formed from a material that is reversibly or elastically deformable, such as an elastomeric material. The elastomeric material the body 18 of the valve unit 10 is formed from, allows for reversible deformation of the first sealing member 15 in relation to the second sealing member 16. This reversible deformation of the first sealing member 15 in relation to the second sealing member 16 allows the valve unit 10 to be held in a state of tension when connected to the fluid supply chamber 20, as described above.

In some embodiments, the body 18 of the valve unit 10 is formed from two or more different elastomeric materials, for example the first sealing member 15 may be formed from a first elastomeric material, and the second sealing member 16 may be formed from a second material. The second material may be elastomeric in nature, however, may instead be a more rigid material.

In some embodiments, the valve unit 10, the body 18, the first sealing member 15, the second sealing member 16, and/or the valve 13 may be made of a Silcone, a heat cured rubber (HCR), a liquid silicone rubber (LSR), or a thermoplastic elastomer material (TPE), such as Thermolast or Santoprene, or any other suitable material.

When the body 18 of the valve unit 10 is formed from one or more materials, the first sealing member 15 may have a first material or structural characteristic, and the second sealing member 16 may have a second material or structural characteristic. In such embodiments, the first material or structural characteristic and the second material or structural characteristic are selected to allow for reversible deformation of the first sealing member 15 with respect to the second sealing member 16. In some embodiments, the first material characteristic and second material characteristic are the same.

In these embodiments, the first material characteristic and second material characteristic may relate to the elastic modulus of the sealing members, and/or the yield stress of the sealing members.

In some embodiments, the first structural characteristic and second structural characteristic may relate to a thickness of the respective sealing members 15, 16.

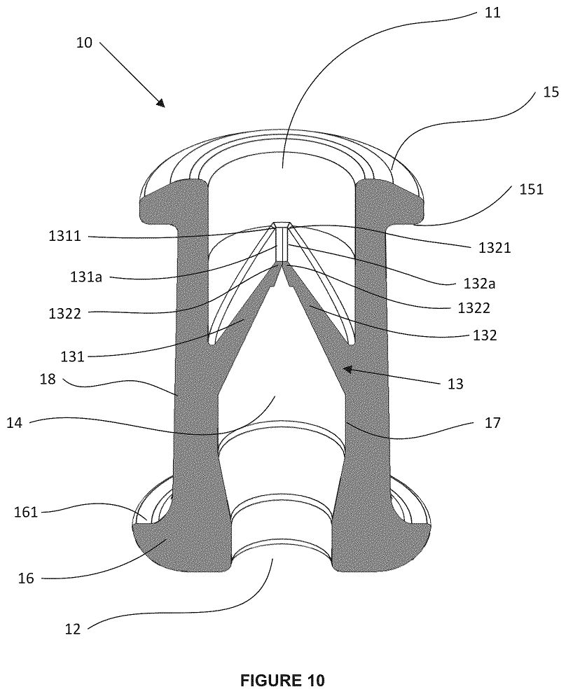

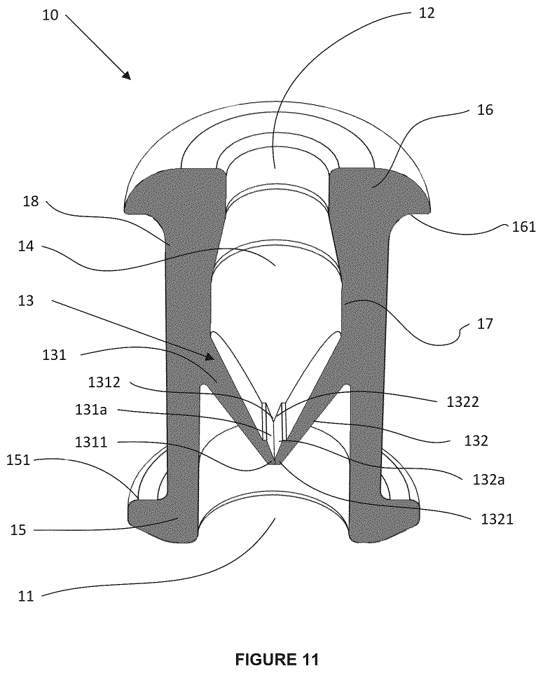

Referring now to FIGS. 10 and 11, the valve 13 of the valve unit 10 further comprises a first flexible flap member 131 and a second flexible flap member 132. The first flexible flap member 131 has a first flap sealing surface 131a extending between two respective lateral ends 1311, 1312 thereof. The second flexible flap member 132 has a second flap sealing surface 132a extending between two respective lateral ends 1321, 1322 thereof.

The first flap sealing surface 131a and the second flap sealing surface 132a are arranged to engage with each other to form a seal to close the valve 13 in a resting state. When closed, the valve 13 prevents fluid from flowing from the first port 11 to the second port 12. The sealing of the first flap sealing surface 131a and the second flap sealing surface 132a prevents any fluid ingress between the sealing surfaces.

When a spike connector is inserted into the second port 12 and through the fluid pathway 14 to be engaged with the valve 13, the first flap sealing surface 131a and the second flap sealing surface 132a disengage from each other to open the valve 13 in an engaged state. When the valve 13 is engaged with the body of the spike connector and the valve unit 10 is in the engaged state, the valve 13 is open and allows fluid entering the first port 11 to flow therethrough towards the second port 12. Fluid from the fluid supply chamber 20 may flow into the fluid passageway 32 of the spike connector and to a conduit which may be attached to the distal end 36 of the spike connector 30.

The body 18 of the valve unit 10 has a fluid passageway 14 extending through it, the fluid passageway 14 extending between the first port 11 and the second port 12. The fluid passageway 14 is defined by the interior wall 17 of the body of the valve unit 10. The interior wall 17 may form part of the fluid passageway 14. Each lateral terminating end of the first flap sealing surface 131a and the second flap sealing surface 132a is integrally formed with or otherwise coupled with an interior wall 17 of the valve unit 10. The first flap sealing surface 131a and the second flap sealing surface 132a may be integrally formed with the interior wall 17 of the valve unit 10, such that the lateral terminating ends form a continuous sealed portion with the interior wall 17.

Additionally, each of the lateral terminating ends 1311, 1312 of the first flap sealing surface 131a may be integrally formed with the corresponding lateral terminating end 1321, 1322 of the second flap sealing surface 132a. As such, the corresponding lateral terminating ends of the first and second flap sealing surfaces may be integrally formed with each other, and with the interior wall 17 of the valve unit body 18. For example, lateral terminating end 1311 of the first flap sealing surface 131a may be integrally formed with lateral terminating end 1321 of the second flap sealing surface 132a where the two lateral terminating ends meet, at the junction with the interior wall 17 of the body 18 of the valve unit 10.

When a spike connector is inserted into the second port 12 and through the fluid pathway 14 and engaged with the valve 13, the first flap sealing surface 131a and the second flap sealing surface 132a engages with at least part of an exterior surface the spike connector. The first flap sealing surface 131a and the second flap sealing surface 132a preferably substantially engage with a part of the exterior surface of the spike connector. In this way, a seal may be formed between the first and second flap sealing surfaces and the portion of the exterior surface of the spike connector. This seal is preferably a watertight seal such that when the valve 13 is in an engaged state, fluid from the fluid supply chamber is prevented from exiting the fluid supply chamber between the exterior of the spike connector and the first and second flap sealing surfaces.

The valve 13 of the valve unit 10 may be formed from an elastomeric material. The elastomeric material may be the same elastomeric material as the body 18 of the valve member 10. As such, the first flexible flap member 131, second flexible flap member 132, and interior wall 17 of the valve unit 10 may be formed from the same material. The first flexible flap member 131 and second flexible flap member 132 may be integrally formed with each other and with the interior wall 17 of the valve unit 10 at the lateral terminating ends.

The elastomeric material that the valve 13 of the valve unit 10 is formed from allows for the first flexible flap member 131 and a second flexible flap member 132 to be reversibly deformable. This reversible deformation of the first flexible flap member 131 and a second flexible flap member 132 allows the flexible flap members of the valve 13 to be engaged with and disengaged with a spike connector, and for the valve 13 to transition between the resting state and the engaged state, as described above.

In some embodiments, the interior wall 17 of the valve unit 10 may have a higher structural integrity than that of the first flexible flap member 131 and/or second flexible flap member 132. The higher structural integrity of the interior wall 17 may refer to a stiffness and/or a thickness and/or a rigidity that is greater than that of the first flexible flap member and/or second flexible flap member.

As each lateral terminating end of the first flap sealing surface 131a and the second flap sealing surface 132a is integrally formed with the interior wall 17 of the valve unit 10, the higher structural integrity of the interior wall 17 in comparison to the flexible flap members provides a high resistance to tear by making it more difficult for a crack to propagate at the termination of the flap sealing surfaces, where the lateral ends meet the interior wall 17, than if the lateral ends where not attached to the interior wall 17.

This higher resistance to tear allows for the valve 13, and specifically the flexible flap members 131, 132, to continually transition between the resting state and the engaged state when a spike connector is inserted into the fluid passageway via the second port 12. This higher resistance to tear of the interior wall 17 prevents the flap sealing surfaces 131a, 132a of the valve 13 from tearing or fatiguing at the termination of the flap sealing surfaces, where the lateral ends meet the interior wall 17.

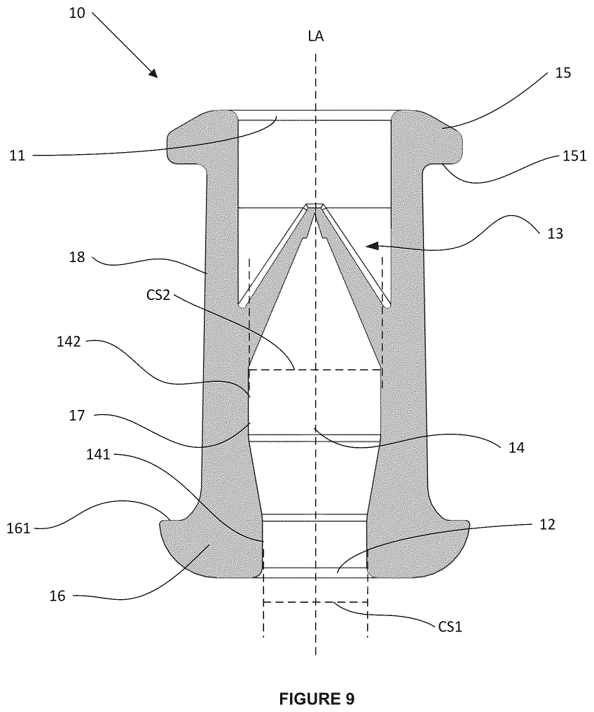

With reference to FIG. 9, the fluid passageway 14 of the valve unit is formed by an interior wall 17 of the body 18 of the valve unit 10 and extending along a longitudinal centre axis LA of the valve unit 10.

The fluid passageway 14 comprises a first section 141 of the interior wall 17 proximal the second port 12 having a first cross section CS1. The first section 141 of the fluid passageway 14 may form the second port 12. The portion of the interior wall 17 defined by the first section 141 is arranged to engage the exterior of the body of a spike connector upon insertion of the spike connector into the fluid passageway 14 to engage the valve 13.

The fluid passageway 14 of the further comprises a second section 142 of the interior wall 17 extending along the longitudinal axis LA from an end of the first section 141 and having a second cross section. The second cross section may gradually increase along the longitudinal axis LA towards the first port 11. After gradually increasing along the longitudinal axis towards the first port 11, the second section 142 may reach a uniform cross section, as shown by CS2. The second section 142 may be represented by a maximum resting cross section of CS2. In alternative embodiments, instead of a gradual increase in cross section after the first section 141 towards the first port 11, there may be a sharp increase in cross section to the second section 142.

As shown, the second cross section is larger than that of the first cross section CS1. The first section 141 and the second section 142 may extend symmetrically around the longitudinal centre axis.

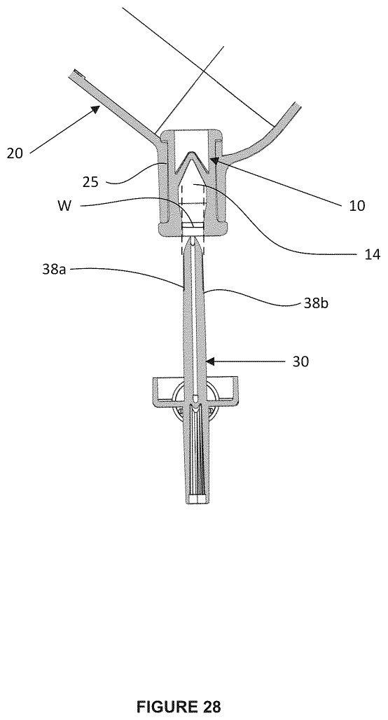

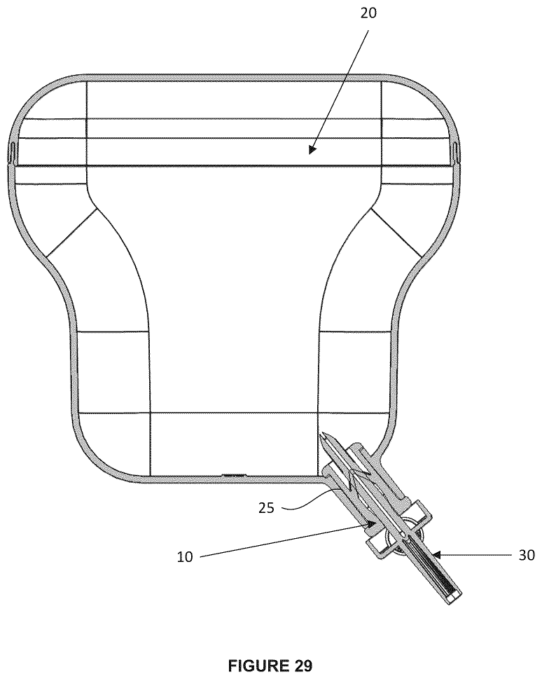

Referring to FIG. 28, the spike connector 30 has an exterior surface and is of a width W or diameter defined between a first exterior surface edge 38a and a second exterior surface edge 38b. The width W or diameter substantially corresponds to the first cross section CS1 of the first portion 141 of the fluid passageway 14.

Due to the substantial similarity between width W of the spike connector, and the first cross section CS1 of the first portion 141 of the fluid passageway 14, the first section 141 of the fluid passageway 14 is adapted to form a seal against the corresponding exterior section of the spike connector 30 upon insertion of the spike connector 30 into the fluid passageway 14 via the second port 12, to engage the valve 13.

As previously discussed with respect to the body 18 of the valve unit 10, the first section 141 of the fluid passageway 14 may also be formed from a material being reversibly or elastically deformable. As such, the reversible or elastic deformability of the first section 141 of the fluid passageway 14 allows for the first section 141 to be in a state of circumferential tension upon insertion of a spike connector into the fluid passageway 14 via the second port 12 to engage the valve 13.

The reversible or elastic deformability of the first section 141, and the substantial similarity between width W of the spike connector and the first cross section CS1 of the first portion 141 of the fluid passageway 14, thereby allows for an interference fit between the circumference of the first portion 141 with the circumference of the exterior surface of the spike connector 30 upon insertion of the spike connector 30 into the fluid passageway 14 to engage the valve 13.

The portion of the interior wall 17 defined by the first section 141 is arranged to engage the exterior of the body of a spike connector upon insertion of the spike connector into the fluid passageway 14 to engage the valve 13. This means that, when the spike connector is inserted into the fluid passageway 14 via the second port 12, a seal is formed between the exterior surface of the spike connector and the first section 141 of the valve unit 10. This seal is therefore formed before the spike connector engages the valve 13. As such, the seal is already present by the time the spike connector engages the valve 13. Therefore, any fluid that may leaks through the valve as it opens upon engagement with the spike connector is prevented from leaking out of the second port 12 during insertion of the spike connector, as it will instead be contained within the second section 142 of the fluid passageway 14.

The fluid passageway 14 increases in diameter along the longitudinal axis LA from the first section 141 that is configured to seal against the exterior surface of the spike connector. As a result, the resistance provided to the insertion or pushing of the spike connector into the valve unit 10 via the second port 12 is substantially constant as the spike port is inserted. As the resistance to insertion or pushing of the spike connector into the valve unit 10 is substantially constant, the user who is inserting or pushing the spike connector into the valve unit 10 are less likely to stop pushing before the spike reaches its fully inserted position whereby it is engaged with the valve 13.

Referring again to FIG. 9, the valve 13 is arranged along the longitudinal axis LA between the first port 11 and the second section 142. The valve 13 is thereby arranged to be positioned downstream along the fluid passageway 14 from the first port 11. As such, the positioning of the valve 13 within the fluid passageway 14 allows for fluid contained at or towards the fluid outlet of the fluid supply chamber to enter the first port 11 and to thereby flow to the fluid passageway of the spike connector.

Referring to FIG. 22, the fluid passageway 14 of the valve unit 10 is formed along a longitudinal centre axis A of the valve unit 10. The valve 13 is arranged in the fluid passageway 14 longitudinally between the first port 11 and second port 12 for controlling the flow of fluid between the first port 11 and second port 12.

The valve 13 is arranged in the fluid passageway 14 along the longitudinal centre axis A between a first point of the longitudinal centre axis A and the second port 12, wherein the first point relates to a point of the longitudinal centre axis intersected by a plane P1 comprising the first sealing surface 151.

Alternatively, the valve 13 is arranged in the fluid passageway 14 along the longitudinal centre axis between a first point of the longitudinal centre axis and the second port 12, wherein the first point is defined by a point of the longitudinal axis intersected by a plane orthogonal to the longitudinal centre axis, which plane intersects a point of the first sealing surface 151 that is closest to the second port.

The present disclosure further relates to an assembly 100 for a respiratory support system that provides humidified respiratory gases. The assembly comprises a valve unit 10 according to any embodiment discussed above, and at least one of the following: a fluid supply chamber 20 for connection to the valve unit 10; a spike connector 30; a supply tube 40 at a first end thereof arranged to connect to the spike connector 30; a humidification chamber 50 for connection to a second end of the supply tube 40; a humidifier 60 comprising the humidification chamber 50 for connection to the second end of the supply tube 40; a gases source 70; a patient interface 80 for connection to the gases source 70 or humidifier 60 by a gases supply tube 90; and a gases supply tube 90 for connection between the gases source 70 and/or humidifier 60 and/or humidification chamber 50 and the patient interface 80.

The humification chamber 50 may comprise an inlet 51 for receiving fluid from the supply tube 40. The humidification chamber 50 may further comprise one or more floats 52 for maintaining a fluid level in the humidification chamber when the supply tube 40 holding fluid is connected to the inlet 51.

The supply tube 40 may be releasably or permanently attached to an inlet 51 of the humidification chamber 50. The spike connector 30 is releasably or permanently attached to the first end of the supply tube 40.

### CLAUSES

Examples of the present disclosure will now be described in the following numbered clauses:
Clause 1. A valve unit for a fluid supply chamber of a respiratory support system that provides humidified respiratory gases, comprising:
   a first port and a second port and a fluid passageway formed therebetween;
   a valve arranged in the fluid passageway for controlling the flow of fluid between the first port and second port;
   a first sealing member having a first sealing surface arranged to seal against an interior surface of the fluid supply chamber proximate the first port when the valve unit is connected to the fluid supply chamber;
   a second sealing member having a second sealing surface arranged to seal against an exterior surface of the fluid supply chamber proximate the second port when the valve unit is connected to the fluid supply chamber.
Clause 2. The valve unit according to clause 1, wherein at least one of the first sealing member, the second sealing member, the valve, and/or the valve unit is reversibly or elastically deformable to put the valve unit under tension when connected to the fluid supply chamber.
Clause 3. The valve unit according to clause 1 or 2, wherein, in a non-connected resting state, the first sealing surface and the second sealing surface are arranged at a closest distance from each other that is smaller than a closest distance between the first sealing surface and the second sealing surface when the valve unit is connected to the fluid supply chamber.
Clause 4. The valve unit according to any one of clauses 1 to 3, wherein the valve in a resting state is closed to prevent fluid entering the first port to flow therethrough towards the second port.
Clause 5. The valve unit (10) according to any one of the preceding clauses, wherein the valve in an engaged state is open to allow fluid entering the first port to flow therethrough towards the second port.
Clause 6. The valve unit according to clause 4, wherein the valve attains its engaged state when a spike connector inserted into the fluid passageway engages the valve.
Clause 7. The valve unit according to any one of the preceding clauses, wherein the valve is a one-way valve or check valve.
Clause 8. The valve unit according to any one of the preceding clauses, wherein the valve is a duck bill valve arranged to open when a spike connector inserted into the fluid passageway engages the duck bill valve.
Clause 9. The valve unit according to any one of the preceding clauses, having a tubular or cylindrical cross-sectional shape.
Clause 10. The valve unit according to any one of the preceding clauses, wherein the first sealing member forms a flange and/or the second sealing member forms a flange.
Clause 11. The valve unit according to any one of the preceding clauses, wherein the first sealing member has a first material or structural characteristic, and the second sealing member has a second material or structural characteristic, wherein the first material or structural characteristic and the second material or structural characteristic are selected to allow for reversible deformation.
Clause 12. The valve unit according to clause 11, wherein the first material characteristic and second material characteristic relates to at least one of elastic modulus and yield stress.
Clause 13. The valve unit according to clause 11 or 12, wherein the first material characteristic and second material characteristic are the same.
Clause 14. The valve unit according to any one of clauses 11 to 13, wherein the first structural characteristic and/or second structural characteristic relate(s) to thickness.
Clause 15. The valve unit according to any one of the preceding clauses, made of an elastomeric material.
Clause 16. The valve unit according to any one of the preceding clauses, wherein the first sealing surface is non-integrally formed with or detachable from the fluid supply chamber.
Clause 17. The valve unit according to any one of the preceding clauses, wherein the first sealing surface is integrally formed with the fluid supply chamber.
Clause 18. The valve unit according to clause 3 or any clause dependent thereon, further comprising a body extending between the first port and second port, wherein the body is made of a material that is reversibly or elastically deformable to allow the closest distance between the first sealing surface and the second sealing surface to increase from that of the non-connected state to that when the valve unit is connected to the fluid supply chamber, so that the valve unit is in tension when the valve unit is connected to the fluid supply chamber.
Clause 19. The valve unit according to any one of the preceding clauses, wherein the valve further comprises:
   a first flexible flap member having a first flap sealing surface extending between two respective lateral ends thereof,
   a second flexible flap member having a second sealing surface extending between two respective lateral ends thereof, wherein the first flap sealing surface and the second sealing surface are arranged to:
      engage each other to form a seal to close the valve in a resting state, and
      disengage from each other to open the valve in an engaged state to allow fluid to flow through the valve,
   wherein each lateral terminating end of the first flap sealing surface and the second flap sealing surface is coupled with an interior wall of the valve unit.
Clause 20. The valve unit according to clause 19, wherein each lateral terminating end of the first flap sealing surface is formed integral with a corresponding lateral terminating end of the second flap sealing surface.
Clause 21. The valve unit according to clause 19 or 20, wherein the first flap sealing surface and the second flap sealing surface in the engaged state engage at least part of an exterior surface the spike connector when the spike connector has been inserted into the fluid passageway to engage the valve.
Clause 22. The valve unit according to any one of clauses 19 to 21, wherein the interior wall forms part of the fluid passageway.
Clause 23. The valve unit according to any one of clauses 19 to 22, wherein the interior wall has a higher structural integrity than that of the first flexible flap member and/or second flexible flap member.
Clause 24. The valve unit according to any one of clauses 19 to 23, wherein the interior wall has a stiffness, thickness, and/or rigidity that is greater than that of the first flexible flap member and/or second flexible flap member.
Clause 25. The valve unit according to any one of clauses 19 to 24, wherein each lateral terminating end of the first flap sealing surface and the second flap sealing surface is formed integral with the interior wall of the valve unit.
Clause 26. The valve unit according to any one of the preceding clauses, wherein the fluid passageway is formed by an interior wall of the valve unit and extending along a longitudinal centre axis of the valve unit and, and the fluid passageway comprising:
   a first section of the interior wall proximal the second port having a first cross section,
   a second section of the interior wall extending along the longitudinal axis from an end of the first section, and having a second cross section that is larger than that of the first cross section,
wherein the first section of the interior wall is arranged to engage a spike connector upon insertion of the spike connector into the fluid passageway to engage the valve.

Clause 27. The valve unit according to clause 26, wherein the valve is arranged between the first port and the second section.
Clause 28. The valve unit according to clause 26 or 27, wherein the second cross section is gradually increasing along the longitudinal axis towards the first port.
Clause 29. The valve unit according to any one of clauses 26 to 28, wherein the first section or second section extend symmetrically around the longitudinal centre axis.
Clause 30. The valve unit according to any one of clauses 26 to 29, wherein the first section is made of a material being reversibly or elastically deformable to put the first section under tension upon insertion of the spike connector into the fluid passageway to engage the valve.
Clause 31. The valve unit according to any one of clauses 26 to 30, wherein the first section forms a seal against a corresponding exterior section of the spike connector upon insertion of the spike connector into the fluid passageway to engage the valve.
Clause 32. The valve unit according to any one of clauses 26 to 31, wherein the first section forms an interference fit against an entire circumference of the spike connector upon insertion of the spike connector into the fluid passageway to engage the valve.
Clause 33. The valve unit according to any one of the preceding clauses, wherein the fluid passageway is formed along a longitudinal centre axis of the valve unit and wherein the valve is arranged in the fluid passageway longitudinally between the first port and second port for controlling the flow of fluid between the first port and second port.
Clause 34. The valve unit according to clause 23, wherein the valve is arranged in the fluid passageway along the longitudinal centre axis between a first point of the longitudinal centre axis and the second port, wherein the first point relates to a point of the longitudinal centre axis intersected by a plane comprising the first sealing surface.
Clause 35. The valve unit according to clause 33 or 34, wherein the valve is arranged in the fluid passageway along the longitudinal centre axis between a first point of the longitudinal centre axis and the second port, wherein the first point is defined by a point of the longitudinal axis intersected by a plane orthogonal to the longitudinal centre axis, which plane intersects a point of the first sealing surface that is closest to the second port.
Clause 36. A valve unit for a fluid supply chamber of a respiratory support system that provides humidified respiratory gases, comprising:
   a first port and a second port and a fluid passageway formed therebetween;
   a valve arranged in the fluid passageway for controlling the flow of fluid between the first port and second port; the valve comprising
      a first flexible flap member having a first flap sealing surface extending between two respective lateral ends thereof,
   a second flexible flap member having a second sealing surface extending between two respective lateral ends thereof, wherein the first flap sealing surface and the second sealing surface are arranged to:
      engage each other to form a seal to close the valve in a resting state, and
      disengage from each other to open the valve in an engaged state to allow fluid to flow through the valve,
wherein each lateral terminating end of the first flap sealing surface and the second flap sealing surface is coupled with an interior wall of the valve unit.
Clause 37. The valve unit according to clause 36, wherein each lateral terminating end of the first flap sealing surface is formed integral with a corresponding lateral terminating end of the second flap sealing surface.
Clause 38. The valve unit according to clause 36 or 37, wherein the first flap sealing surface and the second flap sealing surface in the engaged state engage at least part of an exterior surface of the spike connector when the spike connector has been inserted into the fluid passageway to engage the valve.
Clause 39. The valve unit according to any one of clauses 36 to 38, wherein the interior wall forms part of the fluid passageway.
Clause 40. The valve unit according to any one of clauses 36 to 39, wherein the interior wall has a higher structural integrity than that of the first flexible flap member and/or second flexible flap member.
Clause 41. The valve unit according to any one of clauses 36 to 40, wherein the interior wall has a stiffness, thickness, and/or rigidity that is greater than that of the first flexible flap member and/or second flexible flap member.
Clause 42. The valve unit according to any one of clauses 36 to 41, wherein each lateral terminating end of the first flap sealing surface and the second flap sealing surface is formed integral with the interior wall of the valve unit.
Clause 43. The valve unit according to any one of clauses 36 to 42, further comprising
   a first sealing member having a first sealing surface arranged to seal against an interior surface of the fluid supply chamber proximate the first port when the valve unit is connected to the fluid supply chamber;
   a second sealing member having a second sealing surface arranged to seal against an exterior surface of the fluid supply chamber proximate the second port when the valve unit is connected to the fluid supply chamber.
Clause 44. The valve unit according to clause 43, wherein at least one of the first sealing deformable to put the valve unit under tension when connected to the fluid supply chamber.
Clause 45. The valve unit according to clause 43 or 44, wherein, in a non-connected resting state, the first sealing surface and the second sealing surface are arranged at a closest distance from each other that is smaller than a closest distance between the first sealing surface and the second sealing surface when the valve unit is connected to the fluid supply chamber.
Clause 46. The valve unit according to clause 36 or any clause dependent thereon, wherein the valve in a resting state is closed to prevent fluid entering the first port to flow therethrough towards the second port.
Clause 47. The valve unit according to clause 36 or any clause dependent thereon, wherein the valve in an engaged state is open to allow fluid entering the first port to flow therethrough towards the second port.
Clause 48. The valve unit according to clause 47, wherein the valve attains its engaged state when a spike connector inserted into the fluid passageway engages the valve.
Clause 49. The valve unit according to any one of clauses 36 to 48, wherein the valve is a one-way valve or check valve.
Clause 50. The valve unit according to any one of clauses 36 to 49, wherein the valve is a duck bill valve arranged to open when a spike connector inserted into the fluid passageway engages the duck bill valve.
Clause 51. The valve unit according to any one of clauses 36 to 50, having a tubular or cylindrical cross-sectional shape.
Clause 52. The valve unit according to clause 43 or any clause dependent thereon, wherein the first sealing member forms a flange and/or the second sealing member forms a flange.
Clause 53. The valve unit according to clause 43 or any clause dependent thereon, wherein the first sealing member has a first material or structural characteristic, and the second sealing member has a second material or structural characteristic, wherein the first material or structural characteristic and the second material or structural characteristic are selected to allow for reversible deformation.
Clause 54. The valve unit according to clause 53, wherein the first material characteristic and second material characteristic relates to at least one of elastic modulus and yields stress.
Clause 55. The valve unit according to clause 53 or 54, wherein the first material characteristic and second material characteristic are the same.
Clause 56. The valve unit according to any one of clauses 53 to 55, wherein the first structural characteristic and/or second structural characteristic relate(s) to thickness.
Clause 57. The valve unit according to any one of clauses 36 to 56, made of an elastomeric material.
Clause 58. The valve unit according to clause 36 or any clause dependent thereon, wherein the first sealing surface is non-integrally formed with or detachable from the fluid supply chamber.
Clause 59. The valve unit according to clause 36 or any clause dependent thereon, wherein the first sealing surface is integrally formed with the fluid supply chamber.
Clause 60. The valve unit according to any of clauses 45 or any clause dependent thereon, further comprising a body extending between the first port and second port, wherein the body is made of a material that is reversibly or elastically deformable to allow the closest distance between the first sealing surface and the second sealing surface to increase from that of the non-connected state to that when the valve unit is connected to the fluid supply chamber, so that the valve unit is in tension when the valve unit is connected to the fluid supply chamber.
Clause 61. The valve unit according to clause 60, wherein the body comprises the interior wall.
Clause 62. The valve unit according to clause 36 or any clause dependent thereon, wherein the fluid passageway is formed by the interior wall and extending along a longitudinal centre axis of the valve unit and, the fluid passageway comprising:
   a first section of the interior wall proximal the second port having a first cross section,
   a second section of the interior wall extending along the longitudinal axis from an end of the first section, and having a second cross section that is larger than that of the first cross section,
wherein the first section of the interior wall is arranged to engage a spike connector upon insertion of the spike connector into the fluid passageway to engage the valve.
Clause 63. The valve unit according to clause 62, wherein the valve is arranged between the first port and the second section.
Clause 64. The valve unit according to clause 62 or 63, wherein the second cross section is gradually increasing along the longitudinal axis towards the first port.
Clause 65. The valve unit according to any one of clauses 62 to 64, wherein the first section or second section extend symmetrically around the longitudinal centre axis.
Clause 66. The valve unit according to any one of clauses 62 to 65, wherein the first section is made of a material being reversibly or elastically deformable to put the first section under tension upon insertion of the spike connector into the fluid passageway to engage the valve.
Clause 67. The valve unit according to any one of clauses 62 to 66, wherein the first section forms a seal against a corresponding exterior section of the spike connector upon insertion of the spike connector into the fluid passageway to engage the valve.
Clause 68. The valve unit according to any one of clauses 62 to 67, wherein the first section forms an interference fit against an entire circumference of the spike connector upon insertion of the spike connector into the fluid passageway to engage the valve.
Clause 69. The valve unit according to any one of clauses 36 to 68, wherein the fluid passageway is formed along a longitudinal centre axis of the valve unit and wherein the valve is arranged in the fluid passageway longitudinally between the first port and second port for controlling the flow of fluid between the first port and second port.
Clause 70. The valve unit according to clause 69 when dependent on clause 36 or any clause dependent thereon, wherein the valve is arranged in the fluid passageway along the longitudinal centre axis between a first point of the longitudinal centre axis and the second port, wherein the first point relates to a point of the longitudinal centre axis intersected by a plane comprising the first sealing surface.
Clause 71. The valve unit according to clause 69 when dependent on clause 36 or any clause dependent thereon, wherein the valve is arranged in the fluid passageway along the longitudinal centre axis between a first point of the longitudinal centre axis and the second port, wherein the first point is defined by a point of the longitudinal axis intersected by a plane orthogonal to the longitudinal centre axis, which plane intersects a point of the first sealing surface that is closest to the second port.
Clause 72. A valve unit for a fluid supply chamber of a respiratory support system that provides humidified respiratory gases, comprising:
   a first port and a second port and a fluid passageway formed therebetween;
   a valve arranged in the fluid passageway for controlling the flow of fluid between the first port and second port;
wherein the fluid passageway is formed by an interior wall of the valve unit and extending along a longitudinal centre axis of the valve unit and, and the fluid passageway comprising:
   a first section of the interior wall proximal the second port having a first cross section,
   a second section of the interior wall extending along the longitudinal axis from an end of the first section, and having a second cross section that is larger than that of the first cross section,
wherein the first section of the interior wall is arranged to engage a spike connector upon insertion of the spike connector into the fluid passageway to engage the valve.
Clause 73. The valve unit according to clause 72, wherein the valve is arranged between the first port and the second section.
Clause 74. The valve unit according to clause 72 or 73, wherein the second cross section is gradually increasing along the longitudinal axis towards the first port.
Clause 75. The valve unit according to any one of clauses 72 to 74, wherein the first section or second section extend symmetrically around the longitudinal centre axis.
Clause 76. The valve unit according to any one of clauses 72 to 75, wherein the first section is made of a material being reversibly or elastically deformable to put the first section under tension upon insertion of the spike connector into the fluid passageway to engage the valve.
Clause 77. The valve unit according to any one of clauses 72 to 76, wherein the first section forms a seal against a corresponding exterior section of the spike connector upon insertion of the spike connector into the fluid passageway to engage the valve.
Clause 78. The valve unit according to any one of clauses 72 to 77, wherein the first section forms an interference fit against an entire circumference of the spike connector upon insertion of the spike connector into the fluid passageway to engage the valve.
Clause 79. The valve unit according to any one of clauses 72 to 78, further comprising
a first sealing member having a first sealing surface arranged to seal against an interior surface of the fluid supply chamber proximate the first port when the valve unit is connected to the fluid supply chamber;
a second sealing member having a second sealing surface arranged to seal against an exterior surface of the fluid supply chamber proximate the second port when the valve unit is connected to the fluid supply chamber.
Clause 80. The valve unit according to clause 79, wherein at least one of the first sealing deformable to put the valve unit under tension when connected to the fluid supply chamber.
Clause 81. The valve unit according to clause 79 or 80, wherein, in a non-connected resting state, the first sealing surface and the second sealing surface are arranged at a closest distance from each other that is smaller than a closest distance between the first sealing surface and the second sealing surface when the valve unit is connected to the fluid supply chamber.
Clause 82. The valve unit according to clause 79 or any clause dependent thereon, wherein the valve in a resting state is closed to prevent fluid entering the first port to flow therethrough towards the second port.
Clause 83. The valve unit according to any one of clauses 72 to 79, wherein the valve in an engaged state is open to allow fluid entering the first port to flow therethrough towards the second port.
Clause 84. The valve unit according to clause 83, wherein the valve attains its engaged state when a spike connector inserted into the fluid passageway engages the valve.
Clause 85. The valve unit according to any one of clauses 72 to 84, wherein the valve is a one-way valve or check valve.
Clause 86. The valve unit according to any one of clauses 72 to 85, wherein the valve is a duck bill valve arranged to open when a spike connector inserted into the fluid passageway engages the duck bill valve.
Clause 87. The valve unit according to any one of clauses 72 to 86, having a tubular or cylindrical cross-sectional shape.
Clause 88. The valve unit according to clause 79 or any clause dependent thereon, wherein the first sealing member forms a flange and/or the second sealing member forms a flange.
Clause 89. The valve unit according to clause 79 or any clause dependent thereon, wherein the first sealing member has a first material or structural characteristic, and the second sealing member has a second material or structural characteristic, wherein the first material or structural characteristic and the second material or structural characteristic are selected to allow for reversible deformation.
Clause 90. The valve unit according to clause 89, wherein the first material characteristic and second material characteristic relates to at least one of elastic modulus and yields stress.
Clause 91. The valve unit according to clause 89 or 90, wherein the first material characteristic and second material characteristic are the same.
Clause 92. The valve unit according to any one of clauses 89 to 91, wherein the first structural characteristic and/or second structural characteristic relate(s) to thickness.
Clause 93. The valve unit according to any one of clauses 72 to 92, made of an elastomeric material.
Clause 94. The valve unit according to clause 79 or any clause dependent thereon, wherein the first sealing surface is non-integrally formed with or detachable from the fluid supply chamber.
Clause 95. The valve unit according to clause 79 or any clause dependent thereon, wherein the first sealing surface is integrally formed with the fluid supply chamber.
Clause 96. The valve unit according to clause 81 or any clause dependent thereon, further comprising a body extending between the first port and second port, wherein the body is made of a material that is reversibly or elastically deformable to allow the closest distance between the first sealing surface and the second sealing surface to increase from that of the non-connected state to that when the valve unit is connected to the fluid supply chamber, so that the valve unit is in tension when the valve unit is connected to the fluid supply chamber.
Clause 97. The valve unit according to clause 96, wherein the body comprises the interior wall.
Clause 98. The valve unit according to any one of clauses 72 to 97, wherein the valve further comprises:
   a first flexible flap member having a first flap sealing surface extending between two respective lateral ends thereof,
   a second flexible flap member having a second sealing surface extending between two respective lateral ends thereof, wherein the first flap sealing surface and the second sealing surface are arranged to:
      engage each other to form a seal to close the valve in a resting state, and
      disengage from each other to open the valve in an engaged state to allow fluid to flow through the valve,
   wherein each lateral terminating end of the first flap sealing surface and the second flap sealing surface is coupled with the interior wall of the valve unit.
Clause 99. The valve unit according to clause 98, wherein each lateral terminating end of the first flap sealing surface is formed integral with a corresponding lateral terminating end of the second flap sealing surface.
Clause 100. The valve unit according to clause 98 or 99, wherein the first flap sealing surface and the second flap sealing surface in the engaged state engage at least part of an exterior surface the spike connector when the spike connector has been inserted into the fluid passageway to engage the valve.
Clause 101. The valve unit according to any one of clauses 72 to 100, wherein the interior wall forms part of the fluid passageway.
Clause 102. The valve unit according to any one of clauses 98 to 101, wherein the interior wall has a higher structural integrity than that of the first flexible flap member and/or second flexible flap member.
Clause 103. The valve unit according to any one of clauses 98 to 102, wherein the interior wall has a stiffness, thickness, and/or rigidity that is greater than that of the first flexible flap member and/or second flexible flap member.
Clause 104. The valve unit according to any one of clauses 98 to 103, wherein each lateral terminating end of the first flap sealing surface and the second flap sealing surface is formed integral with the interior wall of the valve unit.
Clause 105. The valve unit according to any one of clauses 72 to 104, wherein the fluid passageway is formed along a longitudinal centre axis of the valve unit and wherein the valve is arranged in the fluid passageway longitudinally between the first port and second port for controlling the flow of fluid between the first port and second port.
Clause 106. The valve unit according to clause 105 when dependent on clause 79 or any clause dependent thereon, wherein the valve is arranged in the fluid passageway along the longitudinal centre axis between a first point of the longitudinal centre axis and the second port, wherein the first point relates to a point of the longitudinal centre axis intersected by a plane comprising the first sealing surface.
Clause 107. The valve unit according to clause 105 when dependent on clause 79 or any clause dependent thereon, wherein the valve is arranged in the fluid passageway along the longitudinal centre axis between a first point of the longitudinal centre axis and the second port, wherein the first point is defined by a point of the longitudinal axis intersected by a plane orthogonal to the longitudinal centre axis, which plane intersects a point of the first sealing surface that is closest to the second port.
Clause 108. An assembly for a respiratory support system that provides humidified respiratory gases, comprising:
   a valve unit according to any one of clauses 1 to 35, 35 to 71, or 72 to 107, and at least one of the following:
      a fluid supply chamber for connection to the valve unit;
      a spike connector;
      a supply tube at a first end thereof arranged to connect to the spike connector;
      a humidification chamber for connection to a second end of the supply tube;
      a humidifier comprising the humidification chamber for connection to the second end of the supply tube;
      a gases source;
      a patient interface for connection to the gases source or humidifier by a gases supply tube; and
      a gases supply tube for connection between the gases source and/or humidifier and/or humidification chamber and the patient interface.
Clause 109. The assembly according to clause 108, wherein the humification chamber comprises an inlet for receiving fluid from the supply tube, and
   one or more floats for maintaining a fluid level in the humidification chamber when the supply tube holding fluid is connected to the inlet.
Clause 110. The assembly according to clause 108 or 109, wherein the supply tub is releasably or permanently attached to an inlet of the humidification chamber.
Clause 111. The assembly according to any one of clauses 108 to 110, wherein the spike connector is releasably or permanently attached to the first end of the supply tube.
Clause 112. A fluid supply chamber assembly for a respiratory support system that provides humidified respiratory gases, comprising
   a valve unit according to any one of clauses 1 to 35, 35 to 71, or 72 to 107, and
   a fluid supply chamber arranged to hold a volume of liquid therein and having a fluid access port, wherein the valve unit is connected to the fluid access port.

1. A valve unit for a fluid supply chamber of a respiratory support system that provides humidified respiratory gases, comprising:
a first port and a second port and a fluid passageway formed therebetween;
a valve arranged in the fluid passageway for controlling the flow of fluid between the first port and second port;
a first sealing member having a first sealing surface arranged to seal against an interior surface of the fluid supply chamber proximate the first port when the valve unit is connected to the fluid supply chamber;
a second sealing member having a second sealing surface arranged to seal against an exterior surface of the fluid supply chamber proximate the second port when the valve unit is connected to the fluid supply chamber.

2. The valve unit according to claim 1, wherein at least one of the first sealing member, the second sealing member, the valve, and/or the valve unit is reversibly or elastically deformable to put the valve unit under tension when connected to the fluid supply chamber.

3. The valve unit according to claim 1 or 2, wherein, in a non-connected resting state, the first sealing surface and the second sealing surface are arranged at a closest distance from each other that is smaller than a closest distance between the first sealing surface and the second sealing surface when the valve unit is connected to the fluid supply chamber.

4. The valve unit according to any one of claims 1 to 3, wherein the valve in a resting state is closed to prevent fluid entering the first port to flow therethrough towards the second port.

5. The valve unit (10) according to any one of the preceding claims, wherein the valve in an engaged state is open to allow fluid entering the first port to flow therethrough towards the second port.

6. The valve unit according to claim 4, wherein the valve attains its engaged state when a spike connector inserted into the fluid passageway engages the valve.

7. The valve unit according to any one of the preceding claims, wherein the valve is a one-way valve, check valve, or a duck bill valve arranged to open when a spike connector inserted into the fluid passageway engages the duck bill valve.

8. The valve unit according to any one of the preceding claims, having a tubular or cylindrical cross-sectional shape.

9. The valve unit according to any one of the preceding claims, wherein the first sealing member forms a flange and/or the second sealing member forms a flange.

10. The valve unit according to any one of the preceding claims, wherein the first sealing member has a first material or structural characteristic, and the second sealing member has a second material or structural characteristic, wherein the first material or structural characteristic and the second material or structural characteristic are selected to allow for reversible deformation.

11. The valve unit according to claim 10, wherein the first material characteristic and second material characteristic relates to at least one of elastic modulus and yield stress.

12. The valve unit according to claim 10 or 11, wherein the first material characteristic and second material characteristic are the same.

13. The valve unit according to any one of claims 10 to 12, wherein the first structural characteristic and/or second structural characteristic relate(s) to thickness of the associated sealing member.

14. The valve unit according to any one of the preceding claims, made of an elastomeric material.

15. The valve unit according to any one of the preceding claims, wherein the first sealing surface is non-integrally formed with or detachable from the fluid supply chamber.

16. The valve unit according to any one of the preceding claims, wherein the first sealing surface is integrally formed with the fluid supply chamber.

17. The valve unit according to claim 3 or any claim dependent thereon, further comprising a body extending between the first port and second port, wherein the body is made of a material that is reversibly or elastically deformable to allow the closest distance between the first sealing surface and the second sealing surface to increase from that of the non-connected state to that when the valve unit is connected to the fluid supply chamber, so that the valve unit is in tension when the valve unit is connected to the fluid supply chamber.

18. The valve unit according to any one of the preceding claims, wherein the valve further comprises:
a first flexible flap member having a first flap sealing surface extending between two respective lateral ends thereof,
a second flexible flap member having a second sealing surface extending between two respective lateral ends thereof, wherein the first flap sealing surface and the second sealing surface are arranged to:
engage each other to form a seal to close the valve in a resting state, and
disengage from each other to open the valve in an engaged state to allow fluid to flow through the valve,
wherein each lateral terminating end of the first flap sealing surface and the second flap sealing surface is coupled with an interior wall of the valve unit.

19. The valve unit according to claim 18, wherein each lateral terminating end of the first flap sealing surface is formed integral with a corresponding lateral terminating end of the second flap sealing surface.

20. The valve unit according to claim 18 or 19, wherein the first flap sealing surface and the second flap sealing surface in the engaged state engage at least part of an exterior surface the spike connector when the spike connector has been inserted into the fluid passageway to engage the valve.

21. The valve unit according to any one of claims 18 to 20, wherein the interior wall forms part of the fluid passageway.

22. The valve unit according to any one of claims 18 to 21, wherein the interior wall has a higher structural integrity than that of the first flexible flap member and/or second flexible flap member.

23. The valve unit according to any one of claims 18 to 22, wherein the interior wall has a stiffness, thickness, and/or rigidity that is greater than that of the first flexible flap member and/or second flexible flap member.

24. The valve unit according to any one of claims 18 to 23, wherein each lateral terminating end of the first flap sealing surface and the second flap sealing surface is formed integral with the interior wall of the valve unit.

25. The valve unit according to any one of the preceding claims, wherein the fluid passageway is formed by an interior wall of the valve unit and extending along a longitudinal centre axis of the valve unit and, and the fluid passageway comprising:
a first section of the interior wall proximal the second port having a first cross section,
a second section of the interior wall extending along the longitudinal axis from an end of the first section, and having a second cross section that is larger than that of the first cross section,
wherein the first section of the interior wall is arranged to engage a spike connector upon insertion of the spike connector into the fluid passageway to engage the valve.

26. The valve unit according to claim 25, wherein the valve is arranged between the first port and the second section.

27. The valve unit according to claim 25 or 26, wherein the second cross section is gradually increasing along the longitudinal axis towards the first port.

28. The valve unit according to any one of claims 25 to 27, wherein the first section or second section extend symmetrically around the longitudinal centre axis.

29. The valve unit according to any one of claims 25 to 28, wherein the first section is made of a material being reversibly or elastically deformable to put the first section under tension upon insertion of the spike connector into the fluid passageway to engage the valve.

30. The valve unit according to any one of claims 25 to 29, wherein the first section forms a seal against a corresponding exterior section of the spike connector upon insertion of the spike connector into the fluid passageway to engage the valve.

31. The valve unit according to any one of claims 25 to 30, wherein the first section forms an interference fit against an entire circumference of the spike connector upon insertion of the spike connector into the fluid passageway to engage the valve.

32. The valve unit according to any one of the preceding claims, wherein the fluid passageway is formed along a longitudinal centre axis of the valve unit and wherein the valve is arranged in the fluid passageway longitudinally between the first port and second port for controlling the flow of fluid between the first port and second port.

33. The valve unit according to claim 22, wherein the valve is arranged in the fluid passageway along the longitudinal centre axis between a first point of the longitudinal centre axis and the second port, wherein the first point relates to a point of the longitudinal centre axis intersected by a plane comprising the first sealing surface.

34. The valve unit according to claim 32 or 33, wherein the valve is arranged in the fluid passageway along the longitudinal centre axis between a first point of the longitudinal centre axis and the second port, wherein the first point is defined by a point of the longitudinal axis intersected by a plane orthogonal to the longitudinal centre axis, which plane intersects a point of the first sealing surface that is closest to the second port.

35. A valve unit for a fluid supply chamber of a respiratory support system that provides humidified respiratory gases, comprising:
a first port and a second port and a fluid passageway formed therebetween;
a valve arranged in the fluid passageway for controlling the flow of fluid between the first port and second port;
wherein the fluid passageway is formed by an interior wall of the valve unit and extending along a longitudinal centre axis of the valve unit and, and the fluid passageway comprising:
a first section of the interior wall proximal the second port having a first cross section,
a second section of the interior wall extending along the longitudinal axis from an end of the first section, and having a second cross section that is larger than that of the first cross section,
wherein the first section of the interior wall is arranged to engage a spike connector upon insertion of the spike connector into the fluid passageway to engage the valve.

36. The valve unit according to claim 35, wherein the valve is arranged between the first port and the second section.

37. The valve unit according to claim 35 or 36, wherein the second cross section is gradually increasing along the longitudinal axis towards the first port.

38. The valve unit according to any one of claims 35 to 36, wherein the first section is made of a material being reversibly or elastically deformable to put the first section under tension upon insertion of the spike connector into the fluid passageway to engage the valve.

39. The valve unit according to any one of claims 35 to 38, wherein the first section forms a seal against a corresponding exterior section of the spike connector upon insertion of the spike connector into the fluid passageway to engage the valve and/or wherein the first section forms an interference fit against an entire circumference of the spike connector upon insertion of the spike connector into the fluid passageway to engage the valve.

40. The valve unit according to any one of claims 35 to 39, wherein the valve further comprises:
a first flexible flap member having a first flap sealing surface extending between two respective lateral ends thereof,
a second flexible flap member having a second sealing surface extending between two respective lateral ends thereof, wherein the first flap sealing surface and the second sealing surface are arranged to:
engage each other to form a seal to close the valve in a resting state, and
disengage from each other to open the valve in an engaged state to allow fluid to flow through the valve,
wherein each lateral terminating end of the first flap sealing surface and the second flap sealing surface is coupled with the interior wall of the valve unit.
